SEQUENCE LISTING (27 sequences)

SEQ ID NO: 1 - protein, 16 aa, synthetic construct
RADARADARADARADA

SEQ ID NO: 2 - protein, 13 aa, synthetic construct
  MOD_RES 1: note = N-term Acetylated
  MOD_RES 13: note = C-term Amidated
IEIKIEIKIEIKI

SEQ ID NO: 3 - protein, 12 aa, synthetic construct
  MOD_RES 1: note = N-term Acetylated
  MOD_RES 12: note = C-term Amidated
QLELQLELQLEL

SEQ ID NO: 4 - protein, 12 aa, synthetic construct
  MOD_RES 1: note = N-term Acetylated
  MOD_RES 12: note = C-term Amidated
KLDKLDKLDKLD

SEQ ID NO: 5 - protein, 16 aa, synthetic construct
KADAKADAKADAKADA

SEQ ID NO: 6 - protein, 8 aa, synthetic construct
RADARADA

SEQ ID NO: 7 - protein, 8 aa, synthetic construct
KADAKADA

SEQ ID NO: 8 - protein, 4 aa, synthetic construct
RADA

SEQ ID NO: 9 - protein, 4 aa, synthetic construct
KADA

SEQ ID NO: 10 - protein, 16 aa, synthetic construct
RAEARAEARAEARAEA

SEQ ID NO: 11 - protein, 4 aa, synthetic construct
RGDS

SEQ ID NO: 12 - protein, 6 aa, synthetic construct
GRGDSP

SEQ ID NO: 13 - protein, 12 aa, synthetic construct
RADARADARADA

SEQ ID NO: 14 - protein, 12 aa, synthetic construct
KADAKADAKADA

SEQ ID NO: 15 - protein, 16 aa, synthetic construct
RGDYRYDYRYDYRGDY

SEQ ID NO: 16 - protein, 16 aa, synthetic construct
RGDFRFDFRFDFRGDF

SEQ ID NO: 17 - protein, 16 aa, synthetic construct
RGDWRWDWRWDWRGDW

SEQ ID NO: 18 - protein, 23 aa, synthetic construct
GRGDIPASSKGGGGSRLLLLLLR

SEQ ID NO: 19 - protein, 16 aa, synthetic construct
RFDARFDARFDARFDA

SEQ ID NO: 20 - protein, 16 aa, synthetic construct
RADFRADFRADFRADF

SEQ ID NO: 21 - protein, 16 aa, synthetic construct
  MOD_RES 1: note = N-term Acetylated
  MOD_RES 16: note = C-term Amidated
RADARADARADARADA

SEQ ID NO: 22 - protein, 4 aa, synthetic construct
RFDA

SEQ ID NO: 23 - protein, 8 aa, synthetic construct
RFDARFDA

SEQ ID NO: 24 - protein, 12 aa, synthetic construct
RFDARFDARFDA

SEQ ID NO: 25 - protein, 4 aa, synthetic construct
RADF

SEQ ID NO: 26 - protein, 8 aa, synthetic construct
RADFRADF

SEQ ID NO: 27 - protein, 12 aa, synthetic construct
RADFRADFRADF

(19) United States
(12) Patent Application Publication
(10) Pub. No.: US 2026/0083817 A1
(43) Pub. Date: Mar. 26, 2026

(54) PEPTIDE COMPOSITIONS AND THEIR USE FOR THE TREATMENT OF SKIN AND MUCOUS MEMBRANE WOUNDS AND IN COSMETIC APPLICATIONS

(71) Applicants: 3-D Matrix, Ltd., Newton, MA (US); Centre national de la recherche scientifique (CNRS), PARIS Cedex 16 (FR)

(72) Inventors: Patricia Rousselle, Lyon (FR); Chloé Laigle, Feyzin (FR); Marie Reveiller Buffier, Fontaines Saint Martin (FR); Sharanya Sankar, Rillieux (FR); Vincent Mortureux, Lyon (FR); Marika G. Rioult, Cambridge, MA (US)

(73) Assignees: 3-D Matrix, Ltd., Newton, MA (US); Centre national de la recherche scientifique (CNRS), PARIS Cedex 16 (FR)

(21) Appl. No.: 19/337,620

(22) Filed: Sep. 23, 2025

Related U.S. Application Data
(60) Provisional application No. 63/697,917, filed on Sep. 23, 2024.

(51) Int. Cl. A61K 38/16; A61K 8/64; A61K 38/07; A61K 38/08; A61K 38/10; A61P 17/02; A61Q 19/00

(52) U.S. Cl. CPC A61K 38/16; A61K 8/64; A61K 38/07; A61K 38/08; A61K 38/10; A61P 17/02; A61Q 19/00

(57) ABSTRACT

The disclosure provides compositions and uses of various RGD-like peptides that interact with laminin-332 and have a minimal sequence of KADA-4 (SEQ ID NO:9), RADA-4 (SEQ ID NO:8), RFDA-4 (SEQ ID NO:22), or RADF-4 (SEQ ID NO:25). The uses include treatment of skin and mucous membrane wounds as well as cosmetic uses of the aforementioned peptides.

### PRIORITY

This application claims priority to U.S. provisional application No. 63/697,917 filed on Sep. 23, 2024.

### SEQUENCE LISTING

The instant application contains an XML Sequence Listing which has been submitted electronically and is hereby incorporated by reference in its entirety. The Sequence Listing, created on Sep. 22, 2025, is named 3DM-24-01-LAM3_US-SequenceListing.xml and is 27,583 bytes in size.

### TECHNICAL FIELD

The technical field relates to use of peptides for medical treatment of wounds in skin and mucous membrane as well as their cosmetic uses.

### BACKGROUND

Self-Assembling Peptides—Self-assembling peptides (“SAPs”) are a type of peptide which assembles spontaneously into highly organized nanostructures when placed in an aqueous environment and a chemical or physical change in surrounding conditions occur. One other well-known structure is a nanofibrous biopolymer structure formed by natural collagen. A class of SAPs relevant to this invention consists of alternating hydrophilic and hydrophobic amino acid residues capable of forming beta-sheets. They autonomously assemble into well-ordered nanostructures in neutral water and can temporarily disassemble into individual molecules when high shearing force is applied to them. SAPs can form a hydrogel (also known as SAP gels) depending on their environment such as pH and/or osmolality; for example, they are capable of forming hydrogel when they are placed in the body where they are exposed to near neutral pH. SAP gels have been previously described as being used for a variety of medical applications, e.g., improved wound healing, inducement of hemostasis; reduction of adhesion in interior tissues, and particularly, in the context of surgery; as temporary tissue-void matrix fillers, facilitating ingrowth of natural tissue into such a void. Particular SAPs are described in U.S. Pat. Nos. 5,670,483; 5,955,343; 9,724,448; 10,596,225 and Int'l Pat. Appln. Pub. WO2014/136081; and foreign equivalents thereof. SAP hydrogels may also be used as a depot for drug delivery, providing controlled release in various applications.

Purastat® is one such commercially available peptide hydrogel available from 3-D Matrix, Ltd. which contains a 16-amino acid polypeptide with a repeating sequence of arginine, alanine, and aspartic acid, specifically, RADARADARADARADA (SEQ ID NO:1) (also referred to as “RADA16” or “RADA-16”). The commercial product contains a terminally modified sequence thereof, specifically, Ac-RADARADARADARADA-NH₂ (SEQ ID NO:21) at a concentration of 2.5% w/v. Other SAPs include but are not limited to, Ac-IEIKIEIKIEIKI-NH₂ (SEQ ID NO:2) (also referred to as “IEIK13” or “IEIK-13”), Ac-QLELQLELQLEL-NH₂ (SEQ ID NO:3) (also referred herein as “QLEL12” or “QLEL-12”), Ac-KLDKLDKLDKLD-NH₂ (SEQ ID NO:4) (also referred herein as “KLD12” or KLD-12″). Yet other SAPs are also well known in the art.

Laminin332—Basement membranes (BMs) are cell-associated sheet-like extracellular matrices covering the basal aspect of all epithelia and endothelia and surrounding muscle, fat, and peripheral nerve cells. BMs are essential for tissue formation in all animals. They provide mechanical stability and barriers between different cell types and are critically involved in cell differentiation, survival, and migration. Laminins are glycoprotein molecules composed of three chains (α, β and γ) bound through disulfide bonds. There are five distinct α subunits, three β subunits, and three γ subunits that combine into at least 15 different laminin isoforms. These isoforms exhibit tissue and development-specific distributions. Recently, the nomenclature for the laminin family was revised to assign three-digit numbers to each laminin isoform. For example, the best studied laminin isoform consisting of α|β1γ1, formerly known as laminin-1 (Sasaki et al., 2004) is now termed laminin-111, while the laminin isoform composed of α3β3γ2, formerly known as laminin-5 (also termed kalinin, epiligrin, ladsin, or nicein), is now called laminin-332. For a more detailed description of laminin-332, see, e.g., Sugawara et al, Exp Dermatol. 2008 June;17 (6): 473-80. doi: 10.1111/j.1600-0625.2008.00721. x. Laminin-332 is expressed in the basal membrane zone (BMZ) of mucous tissues, including the mouth, pharynx, larynx, and trachea. (Patzelt S, Schmidt E. Autoimmunity against laminin 332. Front Immunol. 2023 Aug. 10; 14:1250115. doi: 10.3389/fimmu.2023.1250115. PMID: 37638011; PMCID: PMC10449457.) It is also found in the skin, kidney, lung, and small intestine. Laminin-332 expression increases during wound healing, which affects keratinocyte migration and invasion. The expression of laminin-332 is upregulated during wound healing and has important effects on keratinocyte migration, invasion, and eventually tissue remodeling (D′Alessio, 2008).

While a multitude of methods and compositions exist for treatment of wounds and cosmetic application, there continues to be a need to provide new and improved compositions and methods, particularly for hard-to-treat wounds.

### SUMMARY

The present invention is based at least in part on the discovery that certain self-assembling peptides, shortened versions and analogues thereof, and certain solutions may be advantageous for the treatment of skin and mucous membrane wounds and in cosmetic application. Surprisingly, it was found that of all the various gels tested, only RADA-16 (SEQ ID NO: 1) and KADA-16 (SEQ ID NO:5) interacted with laminin-332 and promoted epidermal repair and regeneration. Furthermore, the binding activity was maintained when the R was replaced by a lysine (K), but was lost when D was replaced by glutamic acid (E). Overall, these results show that KADA-16 (SEQ ID NO:5) is an alternative to RADA-16 (SEQ ID NO:1) for the hydrogel to promote the binding of laminin-332 and that the amino acid D at the third position plays a key role in this interaction. Additionally, it was unexpectedly found that abridged versions of these peptides such RADA-8 (SEQ ID NO:6), KADA-8 (SEQ ID NO:7) and RADA-4 (SEQ ID NO:8), KADA-4 (SEQ ID NO:9) also exhibited similar properties as their full-length 16 amino acid counterparts.

The invention is further based on the findings demonstrating that RADA-16 (SEQ ID NO: 1) promotes faster skin wound closure in vivo, regeneration of the colon and esophageal epithelium, rapid and specific deposition of laminin-332, and promotes migration of epidermal keratinocytes in a laminin-332 dependent manner.

Further investigation of properties of the RADA and KADA peptide sequences, and truncated versions of 1 and 2 repeats of RADA and KADA, were tested for their ability to bind laminin-332. Although these shortened versions of RADA-16 (SEQ ID NO:1) and KADA-16 (SEQ ID NO:5) did not self-assemble into a gel but remained soluble, they retained their ability to bind to laminin-332. This clearly shows that the sequence itself is important for laminin interaction rather than its organization into 3-dimensional matrices. These short peptides, being cheaper and easier to produce, are of obvious interest for the regeneration of epithelia after superficial injuries or for use in the field of cosmetics, which is strongly inspired by the mechanisms of skin regeneration to develop a concept of rejuvenation as an innovative anti-aging strategy. Cosmetic uses of the compositions are also provided, for example, for use after dermabrasion or treatment of superficial wounds. Formulation of the peptides of the invention in lotions and ointments is also contemplated.

Accordingly, the present invention provides novel compositions, comprising one or more peptides interacting with laminin-332 and methods for the treatment of skin and mucous membrane wounds and their cosmetic uses.

In certain embodiments, the composition of the invention contains at least one of the peptides chosen from: consisting of KADA-16 (SEQ ID NO:5); consisting of RADA-8 (SEQ ID NO: 6), consisting of KADA-8 (SEQ ID NO:7), consisting of RADA-4 (SEQ ID NO:8), and consisting of KADA-4 (SEQ ID NO:9).

In some embodiments, the composition comprises only a single peptide comprising the peptide chosen from RADA-4 (SEQ ID NO:8), KADA-4 (SEQ ID NO:9), RADA-8 (SEQ ID NO: 6), KADA-8 (SEQ ID NO:7), and KADA-16 (SEQ ID NO:5).

In certain embodiments, the composition of the invention contains RADA-16 (SEQ ID NO: 1) and one or more peptides chosen from RADA-4 (SEQ ID NO:8), KADA-4 (SEQ ID NO: 9), RADA-8 (SEQ ID NO:6), KADA-8 (SEQ ID NO:7), and KADA-16 (SEQ ID NO:5).

The various combinations of the peptides will be described in detail below. In all of the described embodiments, RADA-16 (SEQ ID NO:1) and its variants can be fully substituted by AC5® (Arch BioSurgery, Framingham, MA) and corresponding truncations and variants thereof.

In preferred embodiments, the composition are sterile so that they may be safely used in various therapeutic or cosmetic applications.

In certain embodiments of the invention, the peptide(s) is/are present in water, or an aqueous buffer, at concentration of 0.1-10% w/v.

The invention also features a pre-filled syringe and a spray device.

In certain embodiments, the invention provides a method of treating a wound in the mucous membranes or wound in the skin by administering the composition of the invention, thereby improving the healing of the wound, and optionally, re-administering the composition until a desired level of healing is achieved. In some embodiments, the methods of treating a wound include methods of accelerating or facilitating wound closure. In some embodiments, the wounds being treated are chronic and/or non-healing.

### BRIEF DESCRIPTION OF THE FIGURES

FIG. 1. RADA-16 (SEQ ID NO:1) supports primary human keratinocytes proliferation and growth in a manner comparable to collagen I. (A) Ki-67 positive cells in proliferating keratinocytes over collagen I or RADA-16 (SEQ ID NO:1) vs. days in culture. Ki-67 expression was determined in normal human keratinocytes (NHKs) seeded on coverslips coated with collagen I or RADA-16 (SEQ ID NO:1) 2.5% w/v after 1, 3, 7, 10 and 13 days of culture as indicated. The percentage of Ki67+ cells out of the total number of cells is shown. Three wells per condition were seeded and 6 non-overlapping fields per well were analyzed when cells were proliferative. The mean value of Ki-67-positive cells in relation to the total cells per field is represented by a dot in the graph (6<n>36). Two independent experiments were performed. Shown are means±SD with *** p<0.001 and **** p<0.0001, determined by the Anova test. (B) NHKs were seeded on 96-well plates containing an increasing amount of 2.5% RADA-16 ((SEQ ID NO:1) 0, 5, 15, or 30 μl of 2.5% w/v). Cell growth was monitored for the indicated time using the Alamar blue reagent.

FIG. 2. RADA-16 (SEQ ID NO:1) supports rapid primary human keratinocytes adhesion in a laminin-332 dependent manner. (A) Adhesion of NHKs to collagen I and RADA-16 (SEQ ID NO:1) 2.5% w/v after 30 minutes and 1 hour of contact. The percentage of adherent cells out of the total seeded cells is shown. Each symbol represents a single well within an experiment. Data from 3 independent experiments are mean±SD, with 9<n>18. (B) Representative phase contrast images of NHKs adherent to collagen I or RADA-16 (SEQ ID NO: 1) 2.5% w/v after 30 minutes, 1 hour or 2 hours of incubation. Scale bar=100 μm. (C) Cell adhesion assay to RADA-16 (SEQ ID NO:1) 2.5% w/v in the presence of the function-blocking anti-laminin-332 antibodies pAb 8039 and mAb BM165 at a concentration of 20 μg/ml and 5 μg/ml respectively or with irrelevant mouse IgG. After 1 hour of contact with the substrates, non-adherent cells were counted. The extent of adhesion in each condition was expressed as the percentage of adhesion to RADA-16 (SEQ ID NO:1) in the absence of antibody. Shown are the means±SD with **** p<0.0001 as determined by the Anova test. Each well is represented by a dot. Data were collected in 3 independent experiments.

FIG. 3. RADA-16 (SEQ ID NO:1) directly and rapidly binds laminin-332 and concentrates it on its surface. Laminin-332 binds directly and rapidly to RADA-16 (SEQ ID NO: 1) and concentrates on its surface over time. (A) Binding of laminin-332 to RADA-16 (SEQ ID NO: 1), purified collagen VII (1 μg/well) and collagen I (10 μg/well) in a 96-well plate ELISA-based protein/protein and protein/hydrogel interaction assay. Laminin-332 bound to the substrates was detected by the fluorescence of a FITC-labeled revealing antibody. Each of the six wells seeded in 3 independent experiments is represented by a symbol. Shown are the means±SD with **** p<0.0001 as determined by the Anova assay. (B) Binding of laminin-332 to RADA-16 (SEQ ID NO: 1) over time. Laminin-332 from NHK conditioned medium was incubated for 5, 10, 30 minutes and 2 hours and the extent of interaction was determined as in (A). Shown are mean values+SD with ** p<0.01 and **** p<0.0001 as determined by the Anova test. (C) Schematic representation of the increasing binding of laminin-332 to RADA-16 (SEQ ID NO:1) over time.

FIG. 4. RADA-16 (SEQ ID NO:1) specifically promotes three-dimensional epidermal wound closure in a laminin-332 and RADA-16 (SEQ ID NO:1) concentration-dependent manner. (A) Influence of RADA-16 (SEQ ID NO:1) on epidermal wound closure. A keratinocyte sheet progressively and efficiently closes a wound covered with RADA-16 (SEQ ID NO: 1) gel using Ibidi, GMbH devices within 3 days. Keratinocyte migration on the top of the gel is inhibited by the presence of an anti-laminin 332 blocking antibody, while it remains unaffected by a non-blocking antibody or by IgG controls. The curves show the evolution of the uninjured gel over time. Each symbol is the average of 6 independent replicates. Shown are the means±SD using data from one experiment with n=6. (B) Effect of RADA-16 (SEQ ID NO:1) stiffness on epidermal wound closure. The Radius in vitro wound closure assay was performed to compare the ability of keratinocytes to close wounds with increasing RADA-16 (SEQ ID NO:1) concentration (1.5%, 2% or 2.5% w/v), resulting in increasing gel stiffness. The curves show the evolution of the unwounded gel over time. Each symbol is the average of 6 replicates in four independent experiments. Shown are mean values+SD with *p<0.05 and ** p<0.01.

FIG. 5. Time-lapse video microscopy analysis of keratinocyte migration with increasing concentration of RADA-16 (SEQ ID NO:1). NHKs were seeded on RADA-16 (SEQ ID NO:1) at 1.5%, 2% and 2.5% w/v concentration and their migration was recorded over 16 hours in 24 well plates. Cells from 3 non-overlapping areas of 3 independent wells were tracked using ImageJ. (A) The images show the movement of cells in each gel concentration condition. Each line corresponds to one cell. (B) The total distance traveled and velocity of NHKs on the different RADA-16 (SEQ ID NO:1) gels were calculated. Shown are means±SD of 3 independent experiments with ** p<0.01, *** p<0.001 and **** p<0.0001.

FIG. 6. The highly specific interaction of laminin-332 with RADA-16 (SEQ ID NO: 1) is maintained when the arginine is replaced by a lysine. The binding of laminin-332 to a series of related and unrelated to RADA-16 (SEQ ID NO:1) self-assembled hydrogels was investigated using the Elisa-based protein/hydrogel interaction assay in 96-well plates (see FIG. 3). Coating efficiency was evaluated by PAGE blue staining. Two mutant forms of RADA-16 (SEQ ID NO: 1) that do not affect gel formation (KADA-16 (SEQ ID NO:5) and RAEA-16 (SEQ ID NO:10) were tested as well as sequence-unrelated hydrogels (IEIK-13 (SEQ ID NO: 2) and KLD-12 (SEQ ID NO:4)). Collagen I was used as a negative control. Each of the six wells tested in 3 independent experiments is represented by a symbol. Shown are the means±SD with **** p<0.0001 as determined by the Anova assay.

FIG. 7. Only gels with the ability to bind laminin-332 promote keratinocyte migration and rapid wound closure. Time-lapse video microscopy analysis of keratinocyte migration performed on the indicated RADA-16 (SEQ ID NO:1)-related and unrelated auto-assembling gels was performed and analyzed using the method described in FIG. 5. (A) The images show the movement of cells in each gel condition tested. Each line corresponds to one cell. (B) The total distance traveled and velocity of the NHKs on the different gels were calculated. Shown are mean values+SD with ** p<0.01, *** p<0.001 and **** p<0.0001. (C) The Ibidi, GmbH wound closure assay described in FIG. 4A was applied to compare the healing-promoting effect of RADA-16 (SEQ ID NO:1) 2.5% w/v with that of IEIK-13 (SEQ ID NO: 2) 1% and 1.3% w/v. The curves show the evolution of the unwounded gel over time. Each symbol is the average of 3 replicates in three independent experiments. Shown are mean values±SD.

FIG. 8. RADA-16 (SEQ ID NO:1) promotes faster onset and progression of re-epithelialization in a porcine model. The effect of RADA-16 (SEQ ID NO:1) on wound healing was investigated in a pig model. Full-thickness wounds were created on the backs of pigs and filled with RADA-16 (SEQ ID NO:1) or a commercialized hydrocolloid gel. The wounds were biopsied at days 2, 7, 14, 21, and 28 after wounding, paraffin-embedded and stained with hematoxylin-cosin. The epidermal wound closure (A), the surface of the regenerating epidermis (B), the average thickness of the new epidermis (C), and the surface of the granulation tissue (D) were quantified with the imageJ software. Shown are mean values+SD with ** p<0.01 and **** p<0.0001.

FIG. 9. Epithelial cells of the colon have a laminin-332-dependent affinity to RADA-16 (SEQ ID NO:1) and preferentially migrate over RADA-16 (SEQ ID NO:1). The binding of laminin-332 produced by human normal colon cells CCD841 (A) and the colon cell line HCT116 (B) to a series of cognate and non-related self-assembled RADA-16 (SEQ ID NO: 1) hydrogels was investigated using the Elisa-based protein/hydrogel interaction assay in 96-well plates (scc FIG. 3). Coating efficiency was evaluated by PAGE blue staining. A mutant form of RADA-16 (SEQ ID NO: 1) that does not affect gel formation (RAEA-16 (SEQ ID NO: 10)) was tested as well as sequence-unrelated hydrogels (IEIK-13 (SEQ ID NO:2) and KLD-12 (SEQ ID NO:4)). Collagen I was used as a negative control. Each of the six wells performed in three independent experiments is represented by a symbol. Shown are the means±SD with **** p<0.0001 as determined by the Anova assay. (C) Time-lapse video microscopy analysis of human CCD841 cell migration on the indicated RADA-16 (SEQ ID NO:1)-related and unrelated auto-assembling gels was performed and analyzed using the method described in FIG. 5. The total distance traveled and the velocity of the cells on the different gels were calculated. Shown are mean values+SD with **** p<0.0001.

FIG. 10. Esophageal epithelial cells have a laminin-332-dependent affinity for RADA-16 (SEQ ID NO:1) and preferentially migrate over RADA-16 (SEQ ID NO:1). (A) The binding of laminin-332 produced by primary human normal esophageal cells (HNES) to a series of related and unrelated self-assembled RADA-16 (SEQ ID NO:1) hydrogels was examined using the Elisa-based protein/hydrogel interaction assay in 96-well plates (see FIG. 3). Coating efficiency was evaluated by PAGE blue staining. A mutant form of RADA-16 (SEQ ID NO: 1) that does not affect gel formation (RAEA-16 (SEQ ID NO:10)) was tested as well as sequence-unrelated hydrogels (IEIK-13 (SEQ ID NO:2) and KLD-12 (SEQ ID NO:4)). Collagen I was used as a negative control. Each of the six wells performed in three independent experiments is represented by a symbol. Shown are the means±SD with **** p<0.0001 as determined by the Anova assay. (C) Time-lapse video microscopy analysis of human CCD841 cell migration on the indicated RADA-16 (SEQ ID NO:1)-related and unrelated auto-assembling gels was performed and analyzed using the method described in FIG. 5. The total distance traveled and the velocity of the cells on the different gels were calculated. Shown are mean values+SD with *p<0.05 and **** p<0.0001.

FIG. 11. RADA-4 (SEQ ID NO:8) and RADA-8 (SEQ ID NO:6) bind laminin-332 in a similar way to their arginine/lysine mutants KADA-4 (SEQ ID NO:9) and KADA-8 (SEQ ID NO:7). Shorter RADA and KADA peptides were tested for their ability to bind laminin-332. 10 μg of the non-gelled soluble peptides RADA-8 (SEQ ID NO:6) and KADA-8 (SEQ ID NO:7) (A) as well as RADA-4 (SEQ ID NO:8) and KADA-4 (SEQ ID NO:9) (B) were immobilized in 96-well plates and laminin-332 from NHK-conditioned medium was incubated for 2 hours. The extent of interaction was determined using the Elisa-based laminin-332/peptide interaction protocol described in FIG. 3. Each of the six wells performed in two independent experiments is represented by a symbol. Shown are the means±SD with **** p<0.0001 as determined by the ANOVA assay.

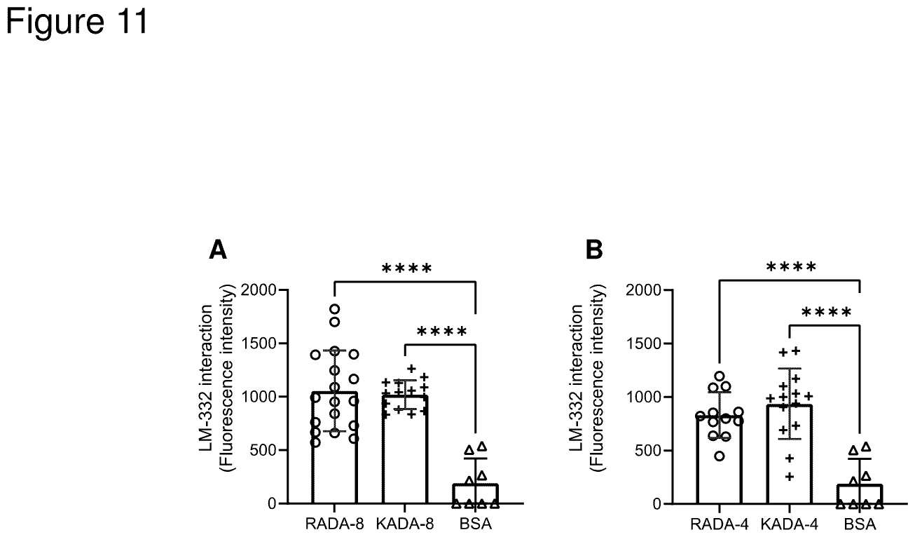

FIG. 12. The interaction between laminin-332 and RADA-16 (SEQ ID NO:1) is highly specific, with the aspartic acid (D) at position 3 playing a critical role. Each amino acid of RADA-16 (SEQ ID NO:1) was systematically substituted with another residue while preserving its self-assembling property. The resulting peptides were immobilized on 96-well plates, and the interaction assay described in FIG. 3 was performed with all mutant variants. Among the tested gels, only KADA-16 (SEQ ID NO:5) and RFDA-16 (SEQ ID NO:19) exhibited binding levels comparable to RADA-16 (SEQ ID NO:1), while RADF-16 (SEQ ID NO: 20) retained partial binding. Notably, the interaction was preserved when arginine (R) was substituted by lysine (K) in KADA-16 (SEQ ID NO:5), but was completely lost when aspartic acid (D) was replaced by glutamic acid (E) in RAEA-16 (SEQ ID NO:10). These findings demonstrate that the binding of laminin-332 to RADA-16 (SEQ ID NO:1) is specific and critically dependent on the aspartic acid residue at the third position, as its substitution abolishes the interaction.

FIG. 13. Behavior of skin fibroblasts in contact with RADA-16 (SEQ ID NO:1) gel. Effect of RGDS (SEQ ID NO:11) peptide on the adhesion of normal human fibroblasts (NHFs) to RADA-16 (SEQ ID NO:1), fibronectin (FN), and collagen I (COL I). Multiwell plates were coated with RADA-16 (SEQ ID NO:1), FN, or bovine serum albumin (BSA).

FIG. 14. Biocompatibility of RADA-16 (SEQ ID NO:1) gels over time. Human NHFs were seeded in 10 μl of 1.5, 2 and 2.5% w/v RADA-16 (SEQ ID NO: 1) and cell growth was monitored for 5 days using the Alamar Blue assay. Cells seeded on plastic served as control (dotted line). The mean values±SD (n=6) are shown. Data are representative of at least 3 independent experiments.

### DETAILED DESCRIPTION

Various conventional full-length SAPs and truncated forms thereof have been tested in this disclosure. These include but are not limited to RADA-16 (SEQ ID NO:1), IEIK-13 (SEQ ID NO: 2), KLD-12 (SEQ ID NO:4), and RAEA-16 (SEQ ID NO:10), and various other variants as described in detail herein.

The present invention is based at least in part on the discovery that certain self-assembling peptides, shortened versions and analogues thereof, and certain solutions may be advantageous for the treatment of skin and mucous membrane wounds and in cosmetic applications. Surprisingly, it was found that of all the gels tested, only RADA-16 (SEQ ID NO: 1), KADA-16 (SEQ ID NO:5), RFDA-16 (SEQ ID NO:19), and RADF-16 (SEQ ID NO:20) interacted with laminin-332 and promoted epidermal repair and regeneration. Furthermore, the binding activity was maintained when the arginine (R) was replaced by a lysine (K), but was lost when aspartic acid (D) was replaced by glutamic acid (E). Overall, these results show that KADA-16 (SEQ ID NO:5), RFDA-16 (SEQ ID NO:19), and RADF-16 (SEQ ID NO:20) are alternatives to RADA-16 (SEQ ID NO:1) for the hydrogel to promote the binding of laminin-332 and that the amino acid D at the third position plays a key role in this interaction. Additionally, it was unexpectedly found that abridged versions of these peptides such RADA-8 (SEQ ID NO:6), KADA-8 (SEQ ID NO:7) and RADA-4 (SEQ ID NO:8), KADA-4 (SEQ ID NO:9) also exhibited similar properties as their full-length 16 amino acid counterparts.

RADA-16 (SEQ ID NO:1), IEIK-13 (SEQ ID NO:2), and KLD-12 (SEQ ID NO:4)—each having unique physical and biochemical properties have been disclosed previously.

PURASTAT® comprises the synthetic peptide Ac-RADARADARADARADA-NH₂ (SEQ ID NO:21), and potentially minor amounts of truncated fragments thereof, and is commercially supplied as a solution at 2.5% wt/vol in water (3-D Matrix, Ltd., Japan). The solution has a pl of 7.2, pK1 of 1.79, and pK2 of 12.58. PURASTAT® exhibits different behavior and properties at different pH values. At pH 2.2, PURASTAT® is a viscous solution. At pH 2.5-4, PURASTAT® forms a semi-rigid, viscous solution. Between pH 4 and 7.5, the self-assembling peptides form a rigid hydrogel. Nanofiber formation is due to hydrophobic and attractive charge-charge interactions.

On the other hand, the SAP IEIK-13 (SEQ ID NO:2) shows different gelation characteristics than RADA-16 (SEQ ID NO:1) when applied as a solution in vitro and in vivo when brought into contact with biological fluids, such as blood, or in vivo-like conditions. SAPs form viscous hydrogels with a nanofibrous matrix in a range of concentrations at about neutral pH. The SAPs disclosed in this application share this and other characteristics despite having different compositions.

Both RADA-16 (SEQ ID NO:1) and IEIK-13 (SEQ ID NO:2) have shown utility in promoting rapid hemostasis when applied to oozing biological tissues and, upon forming a hydrogel matrix integral with wounded tissue, promote normal healing over time rather than scar formation or lack of healing. As reported by Katsuyama et al. (Minimally Invasive Therapy & Allied Technologies 29 (5): 283-292 (2020)), IEIK-13 (SEQ ID NO:2) (called TDM-623 in that reference) forms a stiffer gel (i.e., has a higher storage modulus, G′) compared to RADA-16 (SEQ ID NO:1) (TDM-621 in that reference) when exposed to physiological conditions which correlated with improved hemostasis when the product was applied to liver punch hole injuries in pigs. While reporting statistically significant improvement in hemostasis compared to RADA-16 (SEQ ID NO:1), these authors also reported the absence of inflammatory cell infiltration due to the presence of the RADA-16 (SEQ ID NO:1) or JEIK-13 (SEQ ID NO:2) hydrogel following application of the SAP solutions to the respective wounds in the short-term study. In a more recent study, IEIK-13 (SEQ ID NO:2) (called TDM-623 in that reference) was tested as a hemostatic agent administered endoscopically to oozing wounds created in the walls of the stomach and/or duodenum of pigs with apparent success, although no control group was included (Kubo et al., Endoscopic application of novel, infection-free, advanced hemostatic material: Its usefulness to upper gastrointestinal oozing (2021); doi.org/10.1002/deo2.25). As also found in the previously cited study, hemostasis was achieved in both heparinized and non-heparinized animals. It is noteworthy that, given the acidic pH in the stomach, application of IEIK-13 (SEQ ID NO:2) still resulted in hemostasis of gastric bleeding.

Peptide Concentrations—Rheological properties of peptide compositions as described previously (U.S. Pat. No. 10,654,893, herein incorporated by reference) may be controlled by selection of peptide concentration, for example as may be specifically preferred for a particular indication or use of compositions, through selection and/or adjustment of peptide concentration. For numerous SAPs, composition stiffness has been shown in vitro to increase substantially linearly with peptide concentration.

Any of the peptides of the present invention can be used at various concentrations in solution within a range of (all w/v) from about 0.1% to about 10%; about 0.05% to about 5%, from about 0.05% to about 4%, from about 0.5% to about 4%, from about 0.5% to about 3.5%, from about 0.5% to about 3%, from about 0.5% to about 2.5%, from about 0.5% to about 2%, from about 0.5% to about 1.5%, or about 1%, about 2%, about 2.5%, about 3%, about 4%, or about 5%. As used herein, the percentages of multiple laminin-332 binding peptides are referring to a total concentration of such peptides in solution.

TABLE 1
Non-limiting Exemplary Peptides and Their Combinations

| | |
|---|---|
| Embodiment 1 | KADA-4 (SEQ ID NO: 9), KADA-8 (SEQ ID NO: 7) |
| Embodiment 2 | KADA-4 (SEQ ID NO: 9), KADA-8 (SEQ ID NO: 7), KADA-16 (SEQ ID |
| | NO: 5) |
| Embodiment 3 | KADA-4 (SEQ ID NO: 9), KADA-16 (SEQ ID NO: 5) |
| Embodiment 4 | KADA-4 (SEQ ID NO: 9), RADA-8 (SEQ ID NO: 6) |
| Embodiment 5 | KADA-4 (SEQ ID NO: 9), RADA-4 (SEQ ID NO: 8) |
| Embodiment 6 | KADA-4 (SEQ ID NO: 9), RADA-4 (SEQ ID NO: 8), KADA-8 (SEQ ID |
| | NO: 7) |
| Embodiment 7 | KADA-4 (SEQ ID NO: 9), RADA-4 (SEQ ID NO: 8), RADA-8 (SEQ ID |
| | NO: 6) |
| Embodiment 8 | KADA-4 (SEQ ID NO: 9), RADA-4 (SEQ ID NO: 8), RADA-8 (SEQ ID |
| | NO: 6); RADA-16 (SEQ ID NO: 1) |
| Embodiment 9 | KADA-4 (SEQ ID NO: 9), RADA-4 (SEQ ID NO: 8), RADA-16 (SEQ ID |
| | NO: 1) |
| Embodiment 10 | KADA-4 (SEQ ID NO: 9), KADA-8 (SEQ ID NO: 7), RADA-4 (SEQ ID |
| | NO: 8), RADA-8 (SEQ ID NO: 6), |
| Embodiment 11 | KADA-4 (SEQ ID NO: 9), KADA-8 (SEQ ID NO: 7), RADA-4 (SEQ ID |
| | NO: 8), RADA-8 (SEQ ID NO: 6), RADA-16 (SEQ ID NO: 1) |
| Embodiment 12 | KADA-4 (SEQ ID NO: 9), KADA-8 (SEQ ID NO: 7), RADA-4 (SEQ ID |
| | NO: 8), RADA-8 (SEQ ID NO: 6), KADA-16 (SEQ ID NO: 5) |
| Embodiment 13 | KADA-4 (SEQ ID NO: 9), KADA-8 (SEQ ID NO: 7) |
| Embodiment 14 | KADA-4 (SEQ ID NO: 9), KADA-8 (SEQ ID NO: 7), RADA-8 (SEQ ID |
| | NO: 6), KADA-16 (SEQ ID NO: 5) |
| Embodiment 15 | KADA-4 (SEQ ID NO: 9), RADA-4 (SEQ ID NO: 8); KADA-8 (SEQ ID |
| | NO: 7), RADA-8 (SEQ ID NO: 6) |
| Embodiment 16 | KADA-4 (SEQ ID NO: 9), RADA-4 (SEQ ID NO: 8), KADA-8 (SEQ ID |
| | NO: 7), |
| Embodiment 17 | KADA-4 (SEQ ID NO: 9), RADA-4 (SEQ ID NO: 8), KADA-16 (SEQ ID |
| | NO: 5) |
| Embodiment 18 | KADA-4 (SEQ ID NO: 9), RADA-4 (SEQ ID NO: 8), KADA-8 (SEQ ID |
| | NO: 7), RADA-8 (SEQ ID NO: 6); RADA-16 (SEQ ID NO: 1), KADA-16 |
| | (SEQ ID NO: 5) |
| Embodiment 19 | KADA-4 (SEQ ID NO: 9), RADA-4 (SEQ ID NO: 8), RADA-16 (SEQ ID |
| | NO: 1) |
| Embodiment 20 | KADA-4 (SEQ ID NO: 9), KADA-8 (SEQ ID NO: 7), RADA-4 (SEQ ID |
| | NO: 8), RADA-8 (SEQ ID NO: 6), |
| Embodiment 21 | KADA-4 (SEQ ID NO: 9), KADA-8 (SEQ ID NO: 7), RADA-4 (SEQ ID |
| | NO: 8), RADA-8 (SEQ ID NO: 6), RADA-16 (SEQ ID NO: 1), KADA-16 |
| | (SEQ ID NO: 5) |
| Embodiment 22 | KADA-8 (SEQ ID NO: 7), RADA-8 (SEQ ID NO: 6) |
| Embodiment 23 | KADA-4 (SEQ ID NO: 9), RADA-4 (SEQ ID NO: 8), RADA-12 (SEQ ID |
| | NO: 13) |
| Embodiment 24 | KADA-4 (SEQ ID NO: 9), RADA-12 (SEQ ID NO: 13) |
| Embodiment 25 | KADA-4 (SEQ ID NO: 9), RADA-8 (SEQ ID NO: 6), KADA-12 (SEQ ID |
| | NO: 14) |
| Embodiment 26 | KADA-4 (SEQ ID NO: 9), RADA-4 (SEQ ID NO: 8), KADA-8 (SEQ ID |
| | NO: 7), RADA-8 (SEQ ID NO: 6); KADA-12 (SEQ ID NO: 14), RADA-12 |
| | (SEQ ID NO: 13), RADA-16 (SEQ ID NO: 1), KADA-16 (SEQ ID NO: 5) |
| Embodiment 27 | KADA-4 (SEQ ID NO: 9), RFDA-4 (SEQ ID NO: 22) |
| Embodiment 28 | KADA-8 (SEQ ID NO: 7), RADF-4 (SEQ ID NO: 25) |
| Embodiment 29 | RADA-4 (SEQ ID NO: 8), KADA-4 (SEQ ID NO: 9) |
| Embodiment 30 | RADA-4 (SEQ ID NO: 8), RADF-4 (SEQ ID NO: 25) |

The invention is further based on the findings demonstrating that RADA-16 (SEQ ID NO: 1) promotes faster skin wound closure, regeneration of the colon and esophageal epithelium, rapid and specific deposition of laminin-332, and promotes migration of epidermal keratinocytes in a laminin-332 dependent manner.

In further investigation of properties of the RADA and KADA peptide sequences, truncated versions of 1 and 2 repeats of RADA and KADA were tested for their ability to bind laminin-332. Although the shortened versions of RADA-16 (SEQ ID NO:1) and KADA-16 (SEQ ID NO:5) did not self-assemble into a gel but remained soluble, they retained their ability to bind to laminin-332. This clearly shows that the sequence itself is important for laminin interaction rather than its organization into 3-dimensional matrices. These short peptides, being cheaper and easier to produce, are of interest for the regeneration of epithelia after superficial injuries or for use in the field of cosmetics, which is strongly inspired by the mechanisms of skin regeneration to develop a concept of rejuvenation as an innovative anti-aging strategy. Cosmetic uses of the compositions are also provided, for example, for use after dermabrasion or treatment of superficial wounds. Formulation of the peptides of the invention in lotions and ointments is also contemplated.

Accordingly, the present invention provides novel compositions, comprising one or more peptides interacting with laminin-332 and methods for the treatment of skin and mucous membrane wounds and their cosmetic uses.

In preferred embodiments, the described peptides are administered to a subject, e.g., a human or non-human, in a therapeutically effective amount.

In certain embodiments, the composition of the invention contains at least one of the peptides chosen from: consisting (optionally, essentially) of KADA-16 (SEQ ID NO:5); consisting (optionally, essentially) of RADA-16 (SEQ ID NO:1), consisting (optionally, essentially) of RFDA-16 (SEQ ID NO: 19); consisting (optionally, essentially) of RADF-16 (SEQ ID: 20-12 (SEQ ID NO:13); consisting (optionally, essentially) of RFDA-12 (SEQ ID NO: 24); consisting of RADF-12 (SEQ ID NO:27); consisting (optionally, essentially) of RADA-8 (SEQ ID NO:6), consisting (optionally, essentially) of KADA-8 (SEQ ID NO:7), consisting (optionally, essentially) of RFDA-8 (SEQ ID NO:23); consisting (optionally, essentially) of RADA-4 (SEQ ID NO:8), consisting (optionally, essentially) of KADA-4 (SEQ ID NO:9). In certain embodiments, the composition of the invention consists (optionally, essentially) of any two peptides mentioned in this paragraph, for example, RADA-4 (SEQ ID NO:8) and KADA-4 (SEQ ID NO:9), or KADA-4 (SEQ ID NO:9) and RADF-4 (SEQ ID NO:25). In certain other embodiments, the composition of the invention consists (optionally, essentially) of any three, four or more peptides mentioned in this paragraph, for example, RADA-4 (SEQ ID NO:8), KADA-4 (SEQ ID NO:9), RFDA-4 (SEQ ID NO:22), and RADF-4 (SEQ ID NO:25).

In some embodiments, the composition comprises a single peptide comprising a sequence chosen from RADA-4 (SEQ ID NO:8), KADA-4 (SEQ ID NO:9), RFDA-4 (SEQ ID NO:22), RADF-4 (SEQ ID NO:25), RADA-8 (SEQ ID NO:6), KADA-8 (SEQ ID NO:7), RFDA-8 (SEQ ID NO: 23), RADF-8 (SEQ ID NO:26); KADA-16 (SEQ ID NO:5), RFDA-16 (SEQ ID NO:19), RADF-16 (SEQ ID: 20); RADA-12 (SEQ ID NO: 13), KADA-12 (SEQ ID NO:14), RFDA-12 (SEQ ID NO:24), and RADF-12 (SEQ ID NO:27), or any of the aforementioned peptides fused with so-called “RGD peptides” as described below, or any of the fusions of any of the peptides mentioned in this paragraph. In preferred embodiments, the total length of the final peptide is no more than 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids long. In some of such embodiments, RADA-16 (SEQ ID NO:1) is expressly excluded. In certain preferred embodiments, any of the peptides disclosed herein are N-terminally acetylated and/or C-terminally amidated, which minimizes their degradation in vivo. However, with respect to terminally modified version of RADA16 (SEQ ID NO:21) used for in vitro experiments, no conceptual difference in the behavior is expected as compared to the unmodified version.

The various combinations of the peptides will be described in detail below.

The invention also may feature a pre-filled syringe and a spray device.

In certain embodiments, the invention provides a method of treating a wound in the mucous membranes or wound in the skin by administering the composition of the invention, thereby improving the healing of the wound, and optionally, re-administering the composition until desired level of healing is achieved. In some embodiments, the wound is being treated is chronic or non-healing.

The invention further provides (RADA)n, (KADA)m, (RFDA)x, and/or (RADF)γ peptides fused to each other, or portions of such peptides, whether at the N- or C-terminus, or both, for example, (RADA)n-(KADA)n, (KADA)m-(RADA)n, A-(RADA)n, DA-(RADA)n, ADA-(RADA)n-(KADA) m-KA, (RADA)n-(KADA)n-KAD, (RADA)n-(RFDA)x, ED-(RFDA)x-(KADA) m, etc., wherein n, m, x, y, =0, 1, 2, 3, or 4 indicate the number of the corresponding repeats independently of each other, and wherein n+m+x+y≥z, wherein z is an integer from 1 to 48, for example, 1, 2, 3, 4, 12, 14, 16, 17, 21, 24, 28, 33, 40, 44, 47, and 48.

The invention further provides, as mentioned above, fusion RADA/KADA/RADF/RFDA peptides or mixtures of individual peptides, thereof with “RGD peptides”. Examples of RGD peptides further include:

(SEQ ID NO: 11)
RGDS;
(SEQ ID NO: 15)
RGDYRYDYRYDYRGDY;
(SEQ ID NO: 16)
RGDFRFDFRFDFRGDF;
(SEQ ID NO: 17)
RGDWRWDWRWDWRGDW;
(SEQ ID NO: 18)
GRGDIPASSKGGGGSRLLLLLLR;
(SEQ ID NO: 12)
GRGDSP,

or other shorter or longer RGD-containing peptides or functional fragments thereof, as well as peptides containing such fragments or the full length peptides.

The exact proportions or mixture of peptides may vary and be chosen experimentally, and for example, the proportion, of RADA/KADA/RFDA/RADF/RGD peptides can be determined experimentally, 1:1:1:0, 2:1:1, 1:1:1, 2:2:1; 0:2:1:0, 0:0:1:1, etc. The preferred unit of measurement for mixtures is weight to volume.

The invention further provides a pre-filled syringe, which is prefilled with the composition of the invention. In some embodiments, the composition is sterilized by irradiation, X-ray, e-beam, or gamma-ray as, for example, described in U.S. Pat. No. 11,534,528.

The invention further provides a syringe containing an applicator or spray nozzle, or a spray device, prefilled with the composition of the invention

The invention further provides method of cosmetically and/or therapeutically treating a wound or abrasions by applying one or more compositions of the invention, thereby improving and/or accelerating the healing of a wound, regardless of its location in the body. The composition can be application topically, via an endoscope, or directly to the site of injury, including to wounds that are either chronic or non-healing.

The compositions of the invention can be formulated in a wide variety of ways, whether known in the art or to be developed in the future. These include but are not limited to aqueous or non-aqueous solutions, gels, pastes, suspensions, creams, ointments, mixtures, solids or amorphous formulations, patches, pills, capsules, rinses, implants, coatings, wound dressings, any other compatible formulations regardless of their exact chemical, physical or biological nature so long as they are compatible combinations with other therapeutics and non-therapeutic agents.

In preferred embodiments, the compositions are sterile so that they may be safely used in various therapeutic or cosmetic applications. In preferred embodiments, the compositions have the sterility assurance level (SAL) at least 10⁻⁴, 10⁻⁵, 10⁻⁶, or less.

Sterilization methods are well known in the art. The composition of the invention can be sterilized by, for example, autoclaving, filtration, irradiation (X-ray, e-beam, or gamma-ray as, for example, described in U.S. Pat. No. 11,534,528), or ultra-violent light exposure.

The invention further provides a method of cosmetically/or prophylactically treating intact skin or mucosa, applying to the skin or mucosa single or multiple times, thereby improving skin appearance and/or mitigating its natural or induced deterioration.

### EXAMPLE 1: MATERIALS AND METHODS

Cell culture and antibodies—Three different sources of primary normal human keratinocytes (NHKs) were isolated from newborn foreskins and cultured in keratinocyte growth medium (KBM-2 BulletKit, Lonza Biosciences, Basel, Switzerland) according to a previously published protocol (Boyce and Ham, 1985). Primary normal human fibroblasts (NHFs) were isolated from the same foreskins and cultured in DMEM (Gibco, Life Technologies SAS, Courtaboeuf, France) supplemented with 10% fetal bovine serum (FBS; Hyclone, Cytivia, Saint Germain en Laye, France) according to a previously published protocol (Dos Santos et al., 2016). Human normal esophageal squamous primary cells (HNES) were purchased and cultured according to the manufacturer's instructions (Celprogen, Torrance, USA). Cell lines CCD841 and HCT116 (ATCC) were grown in α-MEM (Lonza) and DMEM (Gibco, Life Technologies SAS, Courtaboeuf, France), respectively, with 10% fetal bovine serum (FBS) at 37° C. and 5% CO2. All cells were incubated at 37° C. and 5% CO2. To obtain cell-conditioned media, FBS- or supplement-free media was left in contact with the cells for 48 hours and centrifuged and filtered before use. The following anti-laminin-332 antibodies were used: mouse monoclonal antibodies 6F12 (Rousselle and Aumailley, 1994) and BM165 (Rousselle et al., 1991); rabbit polyclonal antibodies anti-LM332 pAb 8039 (Roig-Rosello et al, 2024) and pAb 108 (Rousselle Lab). The anti-Ki67 antibody is from Cell signaling (Cliniscience, Nanterre, France). Rabbit pAb against fibronectin was from Dako (Agilent, Les Unis, France) and anti-integrin β4 (clone 3E1, Sigma-Aldrich, Saint Quentin Fallavier, France).

Proteins and peptides—Bovine collagen I was obtained from Symatèse (Chaponost, France) and human plasma fibronectin from Corning (ThermoFisher Scientific). Human LM-332 was purified by affinity chromatography from NHK conditioned medium as previously described (Rousselle & Aumailley, 1994). The NCI domain of human collagen VII was affinity purified using a previously described protocol (Rousselle et al., 1997). Protein concentration was determined with a microprotein assay using bicinchoninic acid (BCA; Pierce, Life Technologies SAS). RADA-16 (SEQ ID NO:1), KADA-16 (SEQ ID NO:5), RFDA-16 (SEQ ID NO:19), RADF-16 (SEQ ID NO:20), IEIK-13 (SEQ ID NO:2) and KLD-12 (SEQ ID NO:4) peptide solutions were produced and provided by 3-D Matrix (3-D Matrix Europe, Caluire, France). RAEA-16 (SEQ ID NO:10) and RADA-4 (SEQ ID NO:8 (SEQ ID NO:11) peptide were purchased from national purchasing center UGAP in France. RGD-FITC was purchased at Eurogentec (Seraing, Belgium).

Cell viability—To examine cell viability on RADA-16 (SEQ ID NO:1) gel, NHKs (n=12, 2×10⁴ cells/well) or NHFs (n=6, 3×10⁴ cells/well) were seeded in 96-well platesuncoated or coated with 5 μL, 15 μL or 30 μL RADA-16 (SEQ ID NO:1) 2.5% w/v gel/well. After 1 hour, the medium was replaced with medium containing 12.5% Alamar Blue solution (Invitrogen, ThermoFisher Scientific, Courtaboeuf, France). To quantify cell viability, measurements were taken immediately and regularly over 24 hours using the Victor X4 spectrophotometer (PerkinElmer S.A.S., Courtaboeuf, France) at 590 nm. A blank with no cells was systematically subtracted for each condition.

Ki-67 immunostaining and quantification of cell proliferation—Immunostaining was performed with NHKs seeded on glass coverslips in 24 well plate coated with bovine collagen I or 2.5% w/v RADA-16 (SEQ ID NO:1) gel NHK (n=3, 2×10⁴ cells/well) and incubated for 1, 3, 7, 10 and 13 days. After washing, cells were fixed with 4% paraformaldehyde/PBS and permeabilized with cold 0.1% Triton X-100 for 5 minutes. Cells were blocked with 1% BSA/PBS for 1 hour and then with 10% goat serum/1% BSA/PBS for 1 hour. Anti-Ki67 pAb was applied for 90 minutes. After washing 3×5 minutes with PBS, the CY3 conjugate was incubated for 30 minutes with fluorescein isothiocyanate phalloidin (FITC; Sigma Aldrich, Saint-Quentin-Fallavier, France) for 30 minutes. Coverslips were washed 3×5 minutes with PBS before mounting with DAPI-containing mounting medium for nuclear staining (Abcam, Cambridge, United Kingdom). The cells were observed with an LSM800 confocal microscope (Carl Zeiss S.A.S., Rucil-Malmaison, France) and images were captured. Using a 20× objective, 6 to 12 non-overlapping areas were imaged. The number of total cells was determined by counting the nuclei and proliferative cells by counting the nuclei expressing Ki-67 using ImageJ software (version 1.53c).

Cell adhesion experiments-RADA-16 (SEQ ID NO:1) 2.5% w/v, collagen I or fibronectin (FN, 5 μg) were applied to the bottom of a multi-well tissue culture plate (96-well, Costar, Corning Life Sciences B.V., Amsterdam, The Netherlands) and then incubated with 5% BSA/PBS for 1 hour. 3 to 5×10⁴ cells were seeded per well and incubated for 30 minutes or 1 hour before being harvested and collected for counting. The cell concentration of the collected supernatants was calculated independently for each well. The number of adherent cells was determined by subtracting the number of non-adherent cells collected from the seeded density and expressed as a percentage of the total number. Adherent cells were photographed using a Zeiss Axiovert 40 microscope equipped with a circular differential interference contrast and coupled to a Coolsnap Fx camera (Roper Scientific, Evry, France).

For cell adhesion inhibition experiments, EDTA 2 mM,anti-laminin-332 antibodies or RGDS (SEQ ID NO: 11) peptide (20 μg/ml) were added at the time of cell seeding. A blank corresponding to wells coated with BSA was automatically subtracted. Each test point was obtained from triplicate wells.

ECM proteins binding assay—To measure the extent of protein interaction, a solution of 0.05% w/v RADA-16 (SEQ ID NO:1), RFDA-16, (SEQ ID NO:19), KADA-16 (SEQ ID NO:5), IEIK-13 (SEQ ID NO:2), KLD-12 (SEQ ID NO:4), RAEA-16-16 (SEQ ID NO:10), and RADF-16 (SEQ ID NO:20), collagen I (5 μg/mL, Symatèse, Chaponost, France), pure collagen VII (10 μg/mL, prepared as described in Rousselle and Aumailley, 1994). The wells were washed 3×5 minutes with 0.02% Tween/PBS before incubation for 1 hour with the anti-laminin-332 mAb 6F12. After washing 3×5 minutes with 0.02% Tween/PBS, a FITC-coupled secondary antibody was applied for 30 minutes. The wells were washed 3×5 minutes with 0.02% Tween/PBS before being read with the Victor X4 PerkinElmer plate reader (Ex/Em wavelengths: 485/535 nm). In some experiments, laminin-332 from fresh NHK-, CCD841-, HCT116- or HNES-conditioned medium was used for the interaction. In this case, a blank corresponding to the wells coated with BSA and a control performed with unconditioned medium were realized and systematically subtracted. At least six wells of each condition were tested and all experiments were repeated 3 times.

Three-dimensional epidermal wound closure assays—NHKs were seeded in 2-well inserts separated by a cell-free gap of 500+/−50 μm (Ibidi culture-insert 2 well, Ibidi GmbH; Cliniscience) at a density of 1.5×10⁴ cells per well. Four hours after seeding, the insert was removed and the cells were washed with PBS. Two μl of 2.5% RADA-16 (SEQ ID NO:1) or other indicated gels were added to the wound, preserving the three-dimensional properties of the gel. The plates were incubated at 37° C. for 4 days. One set of samples was rinsed daily and fixed with 4% paraformaldehyde. Phase contrast mosaics of the samples were imaged with a Nikon Eclipse Ti2-E microscope to visualize cell migration across the gel. The wound area that remained empty and was not colonized by cells was marked in the images using ImageJ software (version 1.53c). The results are expressed as the percentage of uncolonized wound area at each time point relative to the original wound area. The involvement of laminin-332 in wound closure was investigated by blocking the protein with antibodies. Antibodies BM165 and 8039, which block the adhesion promoting function of laminin-332, and antibody 108, which does not block this function, were applied immediately after gel application and renewed after 48 hours of culture. A total mouse IgG was used as a control (Jackson ImmunoResearch, Interchim, Montlucon, France). A series of precise quantitative experiments was performed with the Radius Cell Migration Assay (Cell Biolabs, Cliniscience) in a 24-well plate according to the manufacturer's instructions. NHKs were seeded with 13×10⁴ cells per well, leaving a cell-free area in the center of the well. After performing the washing steps, 0.5 μL of 2.5% w/v RADA-16 (SEQ ID NO:1) or other indicated gels were added to the cell-free center, maintaining the three-dimensional properties of the gels, and the plates were incubated at 37° C. for 6 days. The wells were imaged once per day and mosaics of phase contrast images were taken with the 20× objective of the Nikon Eclipse Ti2-E microscope. Total uncovered areas were quantified using ImageJ software (version 1.53c). The results are expressed as the percentage of uncolonized wound area at each time point relative to the original wound area . . .

Time-lapse video microscopy—To quantify migration on the gels, cells were seeded in transwells for 24-well plates (Falcon, Corning, USA) filled with RADA-16 (SEQ ID NO:1; 1.5%, 2% or 2.5% w/v), RAEA-16 (SEQ ID NO:10; 2.5% w/v), IEIK-13 (SEQ ID NO:2; 1.3% w/v) or KLD-12 (SEQ ID NO:4; 2% w/v) (n=3 and 5×10³ cells/transwell). Phase contrast images were acquired every 15 minutes with a 20× objective of the ZEISS Axio Observer microscope. Migration tracks were created using ImageJ software (version 1.53c), which allows measurement of velocity and total distance traveled by the cells.

Statistical analysis—Data were presented as mean±SD and ANOVA tests were performed using Graph Pad Prism V8. A p-value of p<0.05 was considered statistically significant.

### EXAMPLE 2: RADA-16 (SEQ ID NO:1) PROMOTES THE PROLIFERATION OF EPIDERMAL KERATINOCYTES

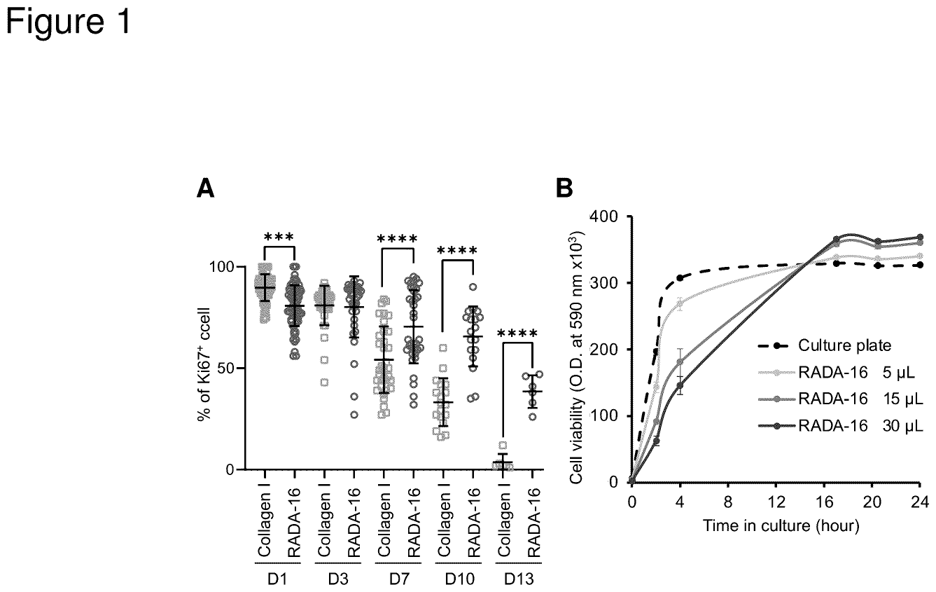

To test the ability of the RADA-16 (SEQ ID NO: 1) gel to promote wound closure, we first examined the behavior of human epidermal keratinocytes that we seeded onto the gel. For this purpose, the proliferation rate of keratinocytes was evaluated using Ki67 staining (FIG. 1A). Collagen I, which is considered the preferential adhesion and migration substrate for keratinocytes during wound healing (Guo et al., 1990), was used as a control. After quantifying Ki67-positive cells over several days of culture, we noted a higher proliferation rate on collagen I on day 1 after seeding, which leveled off on day 3 and then significantly reversed on day 7 with 15% more proliferative cells on the RADA-16 (SEQ ID NO:1) hydrogel. This trend continued on days 10 and 13 where 38% of the cells on RADA-16 (SEQ ID NO:1) were still proliferating while they were no longer proliferating on collagen 1. These results show that the gel supports constant and sustained proliferation until confluence is reached. Because RADA-16 (SEQ ID NO: 1) gel supports continuous and uniform keratinocyte proliferation, we examined the growth and development of a keratinocyte layer seeded on increasing amounts of the gel over time (FIG. 1B). Using Alamar Blue reagent which changes color in response to chemical reduction of the medium by cell growth, we observed progressive cell growth under all conditions tested. The cell culture on plastic was performed as a control. While the cells cultured on a large amount of gel started to grow slowly, the cells cultured on a smaller amount followed the curve we found in the cells plated on plastic; a result that is most likely due to the cells sensing the stiffness of the plastic substrate due to the small amount of gel. As the amount of gel increased, the onset of growth decreased, most likely due to the cells adapting to the thickness and stiffness of the gel. After 12 hours, cell growth under all gel conditions exceeded that of conventional cell culture on plastic. And the more gel was applied, the more cell activity was observed. These results, which show that RADA-16 (SEQ ID NO:1) supports the proliferation and growth of epidermal keratinocytes, make this gel a good candidate for epidermal repair and regeneration.

### EXAMPLE 3: RADA-16 (SEQ ID NO:1) PROMOTES ADHESION OF EPIDERMAL KERATINOCYTES IN A LAMININ-332 DEPENDENT MANNER

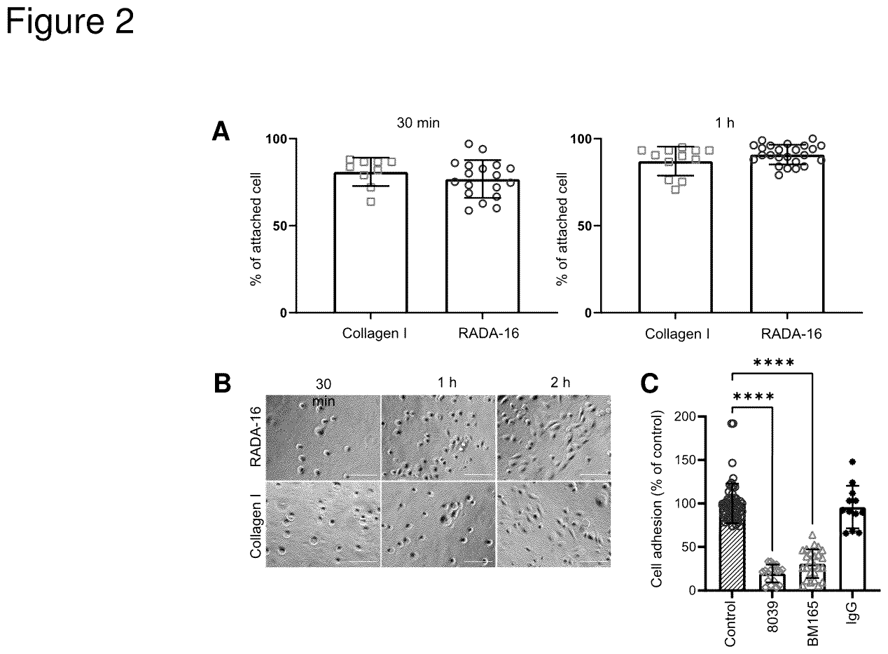

Adhesion and spreading on substrates are essential for keratinocytes, it is a prerequisite for survival, proliferation, migration and differentiation (Watt, 1986). Keratinocyte adhesion is an essential step in wound re-epithelialization as it ensures their survival and allows them to rapidly close the wound to ensure the skin's barrier function (Rousselle et al., 2019). Promoting adhesion in the context of chronic wounds is a major challenge as wound re-epithelialization is essential for healing (Usui et al., 2008). To determine whether RADA-16 (SEQ ID NO:1) supports the rapid adhesion of keratinocytes, a cell adhesion assay was performed (FIG. 2A). Keratinocytes were seeded on RADA-16 (SEQ ID NO:1) and collagen I, and adhesion extent was quantified after 30 minutes and 1 hour. RADA-16 (SEQ ID NO:1) allowed the cells to adhere very quickly, as between 30 minutes and one hour after contact, the percentage of adhered cells was similar to that on collagen 1 (about 80% to 90% of total cells seeded). The keratinocytes not only adhered but also expanded on the gel after one hour to spread completely after two hours, in a manner comparable to that of cells on collagen I (FIG. 2B). This shows that RADA-16 (SEQ ID NO:1) is a favorable substrate for keratinocyte adhesion. In a physiological context and in all epithelia of the body, the protein that mediates epithelial adhesion is the extracellular matrix protein laminin-332, a heterotrimer formed by disulfide-bonded subunits α3β3γ2 encoded by the LAMA3, LAMB3 and LAMC2 genes, respectively (Rousselle and Beck, 2013). Keratinocytes produce large amounts of laminin-332, which they use for their own adhesion via specific receptors (Rousselle et al., 1991; Rousselle and Aumailley, 1994; Tayem et al., 2021). This is an essential step that is often impaired in large or chronic wounds (Fang et al., 2023). To investigate whether keratinocyte adhesion to RADA-16 (SEQ ID NO:1) gel depends on laminin-332 deposition, the cell adhesion assay was performed in the presence of function-blocking anti-laminin-332 antibodies (FIG. 1C). A polyclonal antibody (8039) drastically inhibited cell adhesion and the monoclonal antibody BM165, which targets the integrin binding site in laminin-332, showed the same result with a massive reduction in adhesion. These results show that keratinocytes do not adhere directly to RADA-16 (SEQ ID NO:1), but via a mechanism that is dependent on the deposition of laminin-332 by the cells.

### EXAMPLE 4: KERATINOCYTES FUNCTIONALIZE RADA-16 (SEQ ID NO:1) BY RAPID AND SPECIFIC DEPOSITION OF LAMININ-332

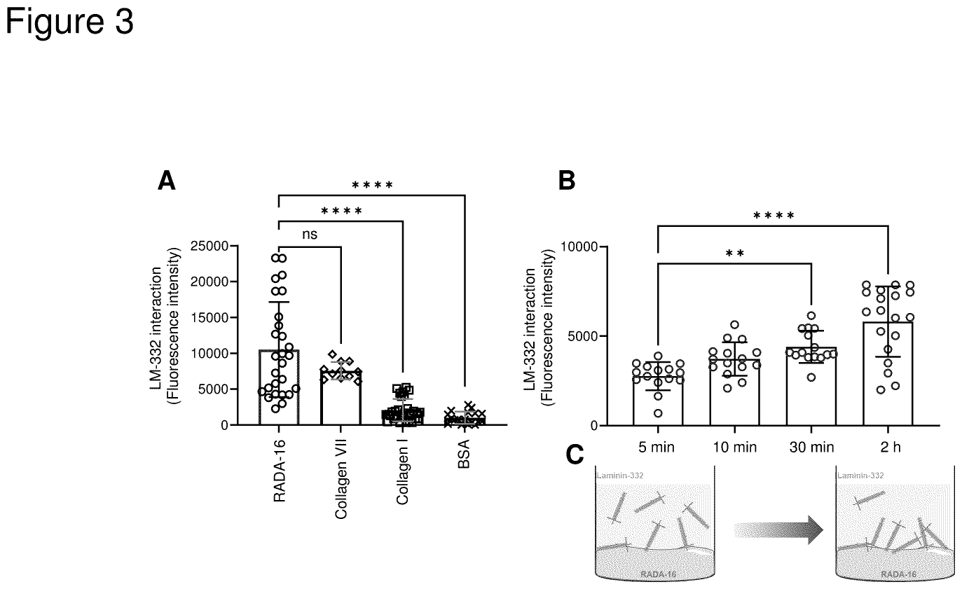

To test the hypothesis of laminin binding to RADA-16 (SEQ ID NO:1), an interaction assay was performed with reference controls such as collagen 1, which is not a partner of laminin-332 (negative control), and collagen VII, which is its molecular partner in vivo (positive control) (Rousselle et al., 1997). The affinity between RADA-16 (SEQ ID NO:1) and laminin-332 was investigated in a protein/peptide interaction assay. As shown in FIG. 3A, purified human laminin-332 interacted strongly with RADA-16 (SEQ ID NO:1), to an extent comparable to that of the positive control collagen VII. As expected, neither collagen 1, nor BSA promoted the interaction. The rapid deposition of laminin-332 is required for an efficient migration process during wound healing. Therefore, the time required for laminin to bind to RADA-16 (SEQ ID NO: 1) was examined using the protein/peptide binding assay as a function of contact time from 5 minutes to 2 hours. As can be seen in FIG. 3B-C, the binding of laminin-332 to RADA-16 (SEQ ID NO: 1) is already important after 5 minutes of contact and increases with time until it reaches almost double the amount after two hours. Unlike many biomaterials that need to be functionalized to enable adhesion and colonization by cells, RADA-16 (SEQ ID NO:1) is functionalized by the cells themselves, which is an important advantage for regenerative medicine as no exogenous recombinant or native human or animal protein needs to be added to the device.

### EXAMPLE 5: RADA-16 (SEQ ID NO:1) PROMOTES MIGRATION OF EPIDERMAL KERATINOCYTES IN A LAMININ-332 DEPENDENT MANNER

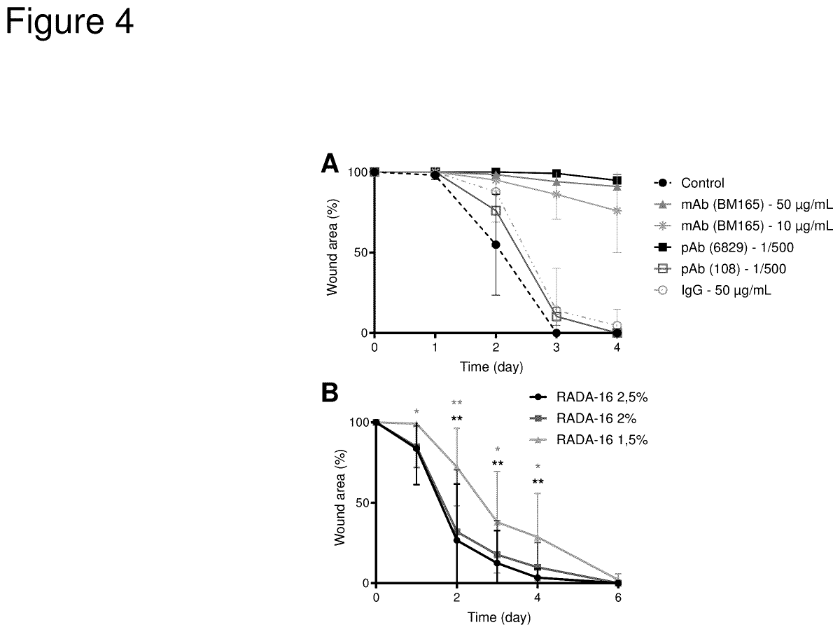

To close a wound, the migration of keratinocytes is essential, and laminin-332 was shown to participate in this process (Nguyen et al., 2000; Décline and Rousselle, 2001; Aragona et al., 2017). As this depends on the production of laminin-332 by the cells, we investigated the ability of the gel to promote the collective migration of epidermal cells in our laboratory-developed 3-dimensional in vitro wound closure assay (FIG. 4A). When keratinocyte sheets were used to close a wound covered with the RADA-16 (SEQ ID NO:1) gel, they gradually and efficiently covered the gel by migrating from the edges and completely closed the wound within three days. Blocking cell interaction with laminin-332 by various function-blocking antibodies completely abolished cell migration and wound closure, whereas this was not affected by a non-blocking antibody or by IgG controls. This result show that the migration process triggered by RADA-16 (SEQ ID NO:1) is based on its ability to recruit laminin-332.

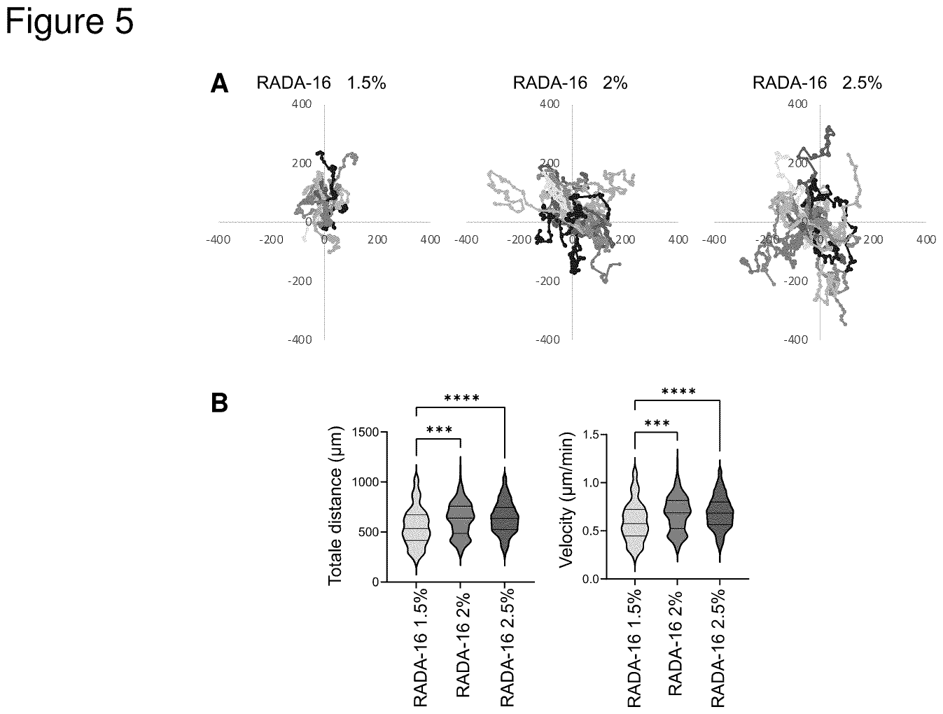

To further analyze the migration-promoting properties of RADA-16 (SEQ ID NO:1), different concentrations of the gel were tested. We found that wound closure was significantly improved when using RADA-16 (SEQ ID NO:1) gels at concentrations of 2% and 2.5% w/v compared to the softer 1.5% w/v gel (FIG. 4B). On day 2, the wound was closed at 73% with the 2% and 2.5% gels, compared to only 28% with the 1.5% gel. The increase in the RADA-16 (SEQ ID NO: 1) peptide concentration not only leads to an increase in the affinity for laminin-332, but also improves the stiffness of the gel. This result was confirmed by a time-lapse video microscopy experiment in which the behavior of the keratinocytes plated on the different gels was recorded over time. As shown by the migration tracks (FIG. 5A), the cells migrated on the different gels, but with much greater efficiency on RADA-16 (SEQ ID NO: 1) at 2% and 2.5% w/v. The higher the RADA-16 (SEQ ID NO: 1) concentration, the more cells migrated. This was confirmed by quantitative analysis of migration parameters such as distance traveled and speed, that were significantly higher on RADA-16 (SEQ ID NO:1) 2% and 2.5% w/v compared to the 1.5% w/v gel (FIG. 5B).

### EXAMPLE 6: THE HIGHLY SPECIFIC INTERACTION OF LAMININ-332 WITH RADA-16 (SEQ ID NO:1) IS MAINTAINED WHEN THE AMINO-ACID ARGININE (R) IS REPLACED BY LYSINE (K)

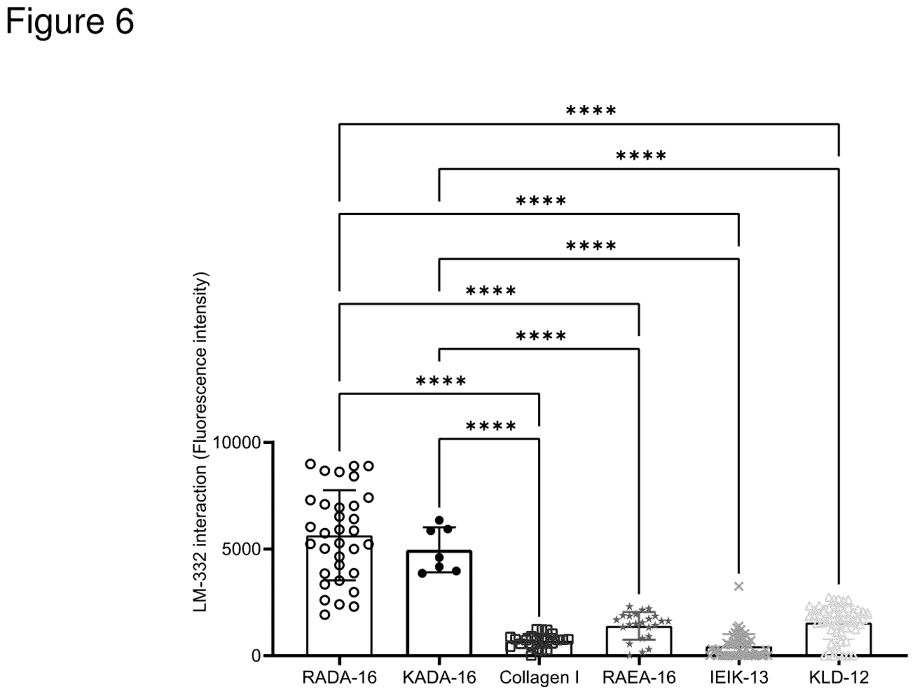

To ensure that the interaction of laminin with RADA-16 (SEQ ID NO:1) is specific and not the result of simple non-specific fixation on the gel, a series of self-assembling gels were tested (FIG. 6). Targeted mutagenesis was also performed to understand the mechanism of the interaction. The two charged amino acids arginine (R-positively charged) and aspartic acid (D-negatively charged) were mutated in such a way that the self-assembling properties of the gel were retained. Collagen I was used as a negative control. The results of the laminin-332 binding assay showed that of all the gels tested, only RADA-16 (SEQ ID NO:1) and KADA-16 (SEQ ID NO: 5) interacted with laminin-332. Interestingly, the binding activity was maintained when the R was replaced by a lysine (K), but was lost when D was replaced by glutamic acid (E). Overall, these results show that KADA-16 (SEQ ID NO:5) is an alternative to RADA-16 (SEQ ID NO:1) for the hydrogel to promote the binding of laminin-332 and that the amino acid D at the third position plays a key role in this interaction.

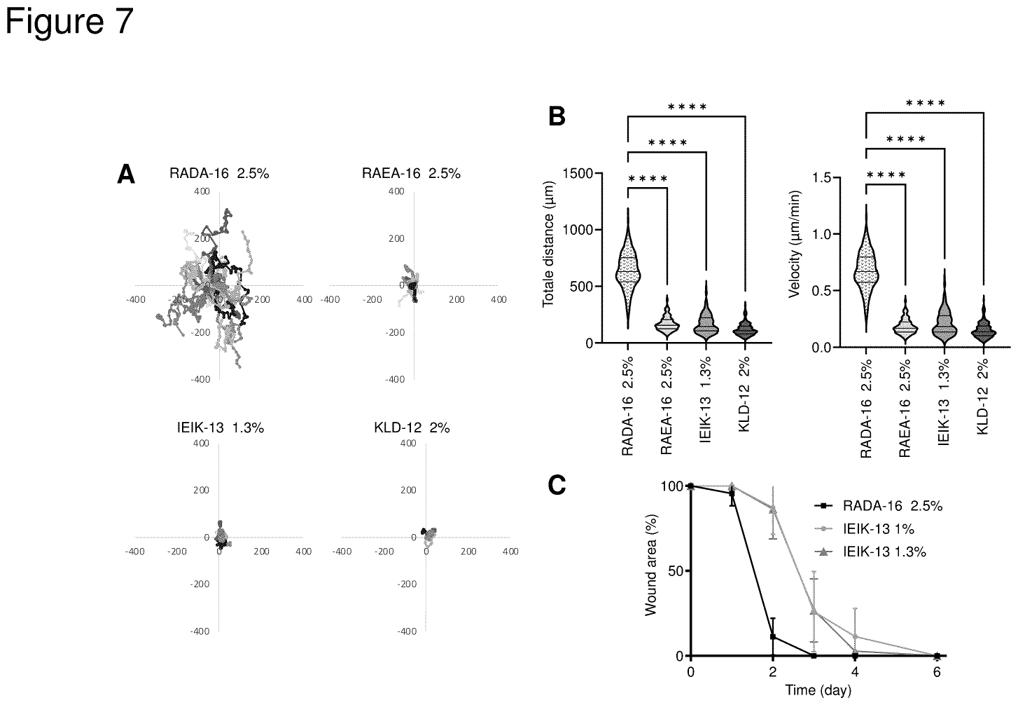

To confirm these results, our video-microscopic time-lapse experiments with these gels show that only RADA-16 (SEQ ID NO: 1) induces keratinocyte migration, while the cells on the RAEA-16 (SEQ ID NO:10), IEIK-13 (SEQ ID NO:2), and KLD-12 (SEQ ID NO:4) gels remained immobile. This property is confirmed by the wound closure test, which shows a clear and significant advantage of the RADA-16 (SEQ ID NO:1) gel over the other auto-assembling peptide-based hydrogels tested. Two days after wounding, around 90% of the wounds were already closed when treated with RADA-16 (SEQ ID NO:1) compared to 14% with IEIK-13 (SEQ ID NO:2) (FIG. 7C).

### EXAMPLE 7: RADA-16 (SEQ ID NO:1) PROMOTES SKIN WOUND CLOSURE IN VIVO

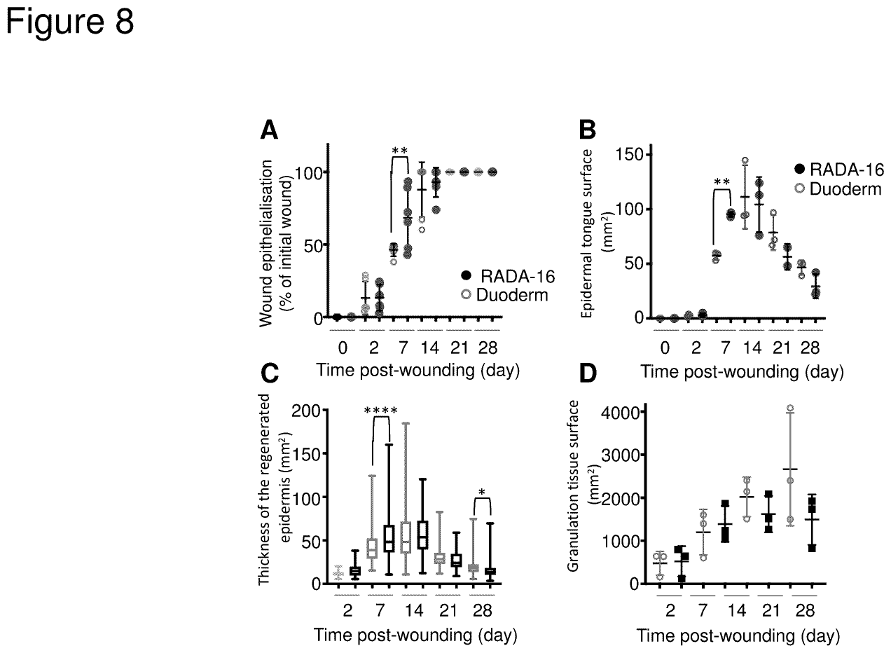

In order to see whether the results obtained in vitro are transferable in vivo, a study was carried out on a pig model in which the effect of RADA-16 (SEQ ID NO: 1) gel was compared with a hydrocolloid gel from the market. Wound closure of punch biopsy full-thickness wounds was followed for 28 days and assessed by hematoxylin and cosin staining of tissue sections at various time points during the healing process. Imaging quantification showed that the wound was already 70% re-epithelialized seven days after treatment, compared to 46% in the control group (FIG. 8A). The regenerated epidermal area was up to 95 mm² thick in the wounds treated with RADA-16 (SEQ ID NO:1), compared to only 60 mm² in the control group, demonstrating the superior proliferative state of the epidermal tongue on RADA-16 (SEQ ID NO:1) (FIG. 8B). At the time of wound resolution, the repaired skin has a significantly thinner epidermis, indicating an earlier termination of regeneration and return to normal epidermis (FIG. 8C, 8D). Finally, measurement of the surface area of the granulation tissue over time revealed that in the RADA-16 (SEQ ID NO: 1) treated wounds, the maximum surface area found at D21 decreased slightly at D28 compared to the granulation tissue of the control wounds, which continued to be of a greater size. This earlier resolution of the granulation tissue is most likely related to the effective re-epithelialization process seen with RADA-16 (SEQ ID NO:1) hydrogel. Thus, the RADA-16 (SEQ ID NO:1) gel showed a preclinical advantage in the initiation, progression and completion of re-epithelialization with positive effects on the dermal granulation tissue resolution.

### EXAMPLE 8: USE OF THE LAMININ-BINDING FUNCTION OF RADA-16 (SEQ ID NO:1) FOR THE REGENERATION OF THE COLON AND ESOPHAGEAL EPITHELIUM

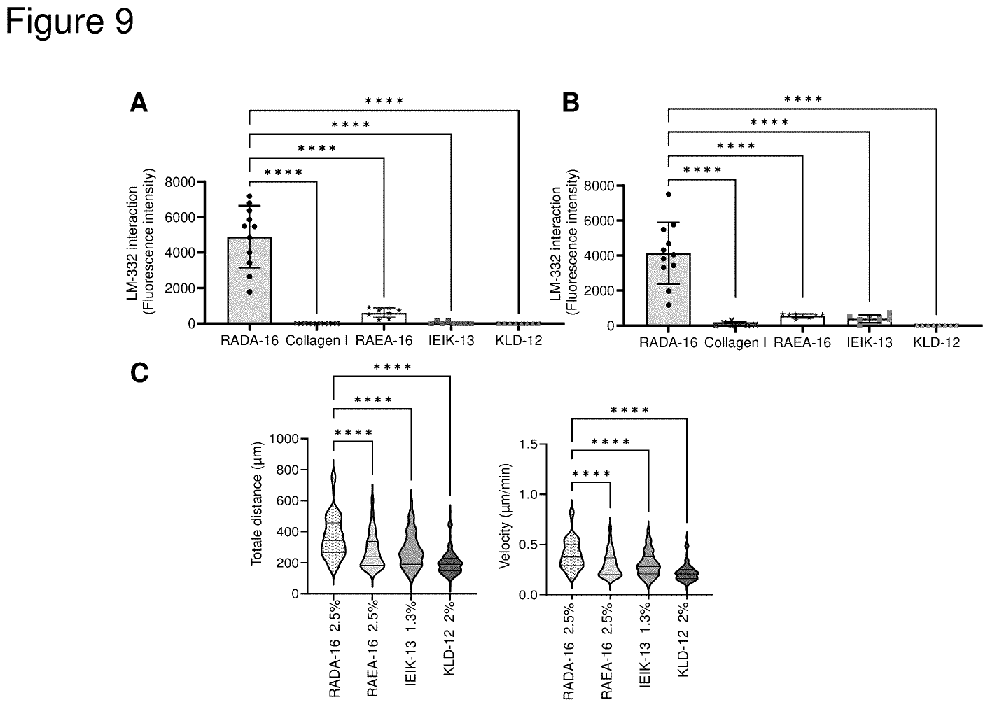

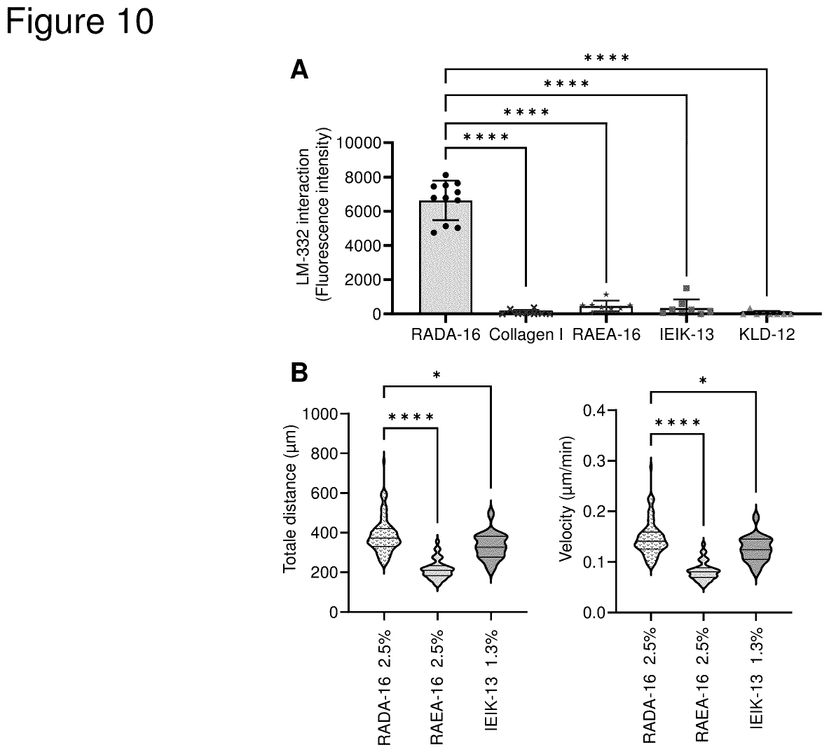

Laminin-332 is expressed in all basement membranes underlying epithelia of the body that have a protective or secretory function, such as the mucosa (Sasaki et al., 2004). As expected, the ability of RADA-16 (SEQ ID NO:1) to bind laminin-332 produced by cells of the colon (Simon-Assmann et al., 1998; Bouatrouss et al., 2000; Dave et al., 2004) and esophagus (Dave et al., 2004; Xue et al., 2011; Evans et al., 2013) is specific and similar to that of keratinocytes of the epidermis, since identical laminin-332 is expressed in these tissues (FIGS. 9A and 10A). In addition, the migration capacity of colon and esophageal cells on the RADA-16 (SEQ ID NO:1) gel was found to be significantly higher compared to other self-assembling gels, as shown by our time-lapse video microscopy experiments (FIGS. 10B-C and 11B-C). This shows that the pro-regenerative properties of RADA-16 (SEQ ID NO: 1) demonstrated for epidermal keratinocytes can also be transferred to other epithelial tissues.

### EXAMPLE 9: USE OF RADA-4 (SEQ ID NO:8) OR KADA-4 (SEQ ID NO:9) AND RADA-8 (SEQ ID NO:6) OR KADA-8 (SEQ ID NO:7) FOR SUPERFICIAL INJURIES AND REJUVENATION TARGETED COSMETIC APPLICATION

To further investigate the properties of the RADA and KADA peptide sequences, we prepared truncated versions of 1 and 2 repeats of RADA and KADA to test their ability to bind laminin-332. Although these shortened versions of RADA-16 (SEQ ID NO: 1) and KADA-16 (SEQ ID NO:5) did not self-assemble into a gel but remained soluble, they retained their ability to bind to laminin-332. This clearly shows that the sequence itself is important for laminin interaction rather than its organization into 3-dimensional matrices. These short peptides are of obvious interest for the regeneration of epithelia after superficial injuries or for use in the field of cosmetics, which is strongly inspired by the mechanisms of skin regeneration to develop a concept of rejuvenation as an innovative anti-aging strategy.

### EXAMPLE 10: THE BINDING OF LAMININ-332 TO RADA-16 (SEQ ID NO:1) IS HIGHLY SPECIFIC, WITH THE ASPARTIC ACID (D) PLAYING A CRITICAL ROLE

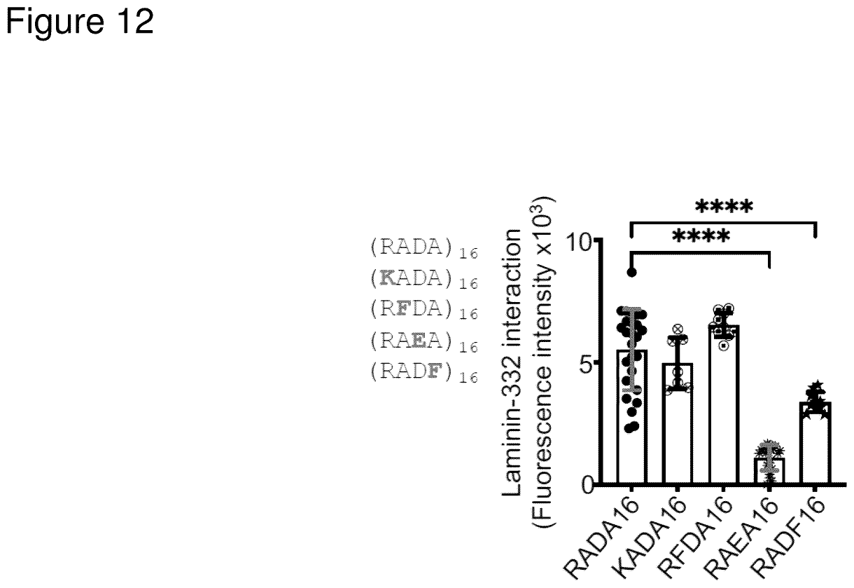

To define the minimal binding site for laminin (LM)-332, a series of mutated peptides were generated and evaluated in the LM-332 binding assay. Among the gels tested, only KADA-16 (SEQ ID NO:5) and RFDA-16 (SEQ ID NO:19) bound LM-332 with efficiency comparable to RADA-16 (SEQ ID NO:1), whereas RADF-16 (SEQ ID NO:20) displayed partial interaction (FIG. 12). Notably, replacing aspartic acid (D) with glutamic acid (E) completely abolished binding, as observed with RAEA-16 (SEQ ID NO:10). These results demonstrate that LM-332 binding to RADA-16 (SEQ ID NO:1) is highly specific and critically dependent on the aspartic acid at the third residue. This specificity supports a molecular mechanism by which RADA-16 (SEQ ID NO:1) selectively recruits the keratinocyte adhesion protein LM-332, a property likely contributing to its ability to mimic extracellular matrix components and promote cellular adhesion during wound healing.

### EXAMPLE 11: RADA-16 (SEQ ID NO:1) SUPPORTS THE ADHESION AND GROWTH OF DERMAL FIBROBLASTS

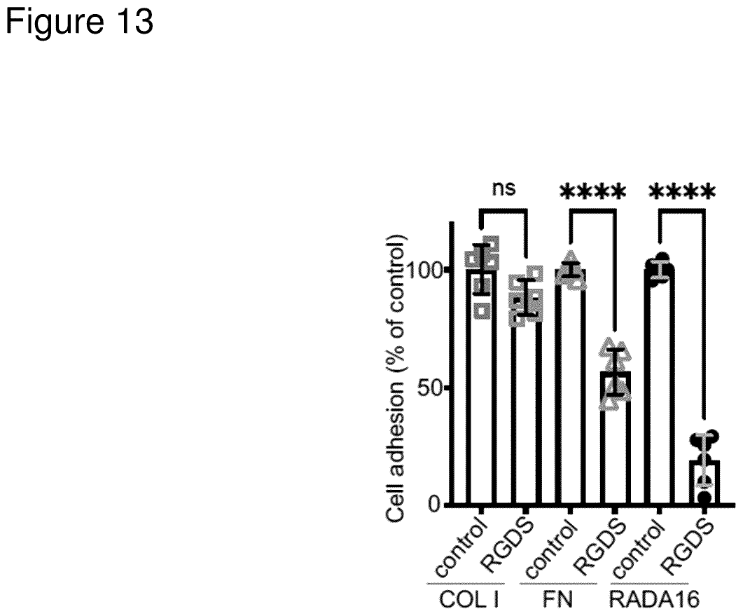

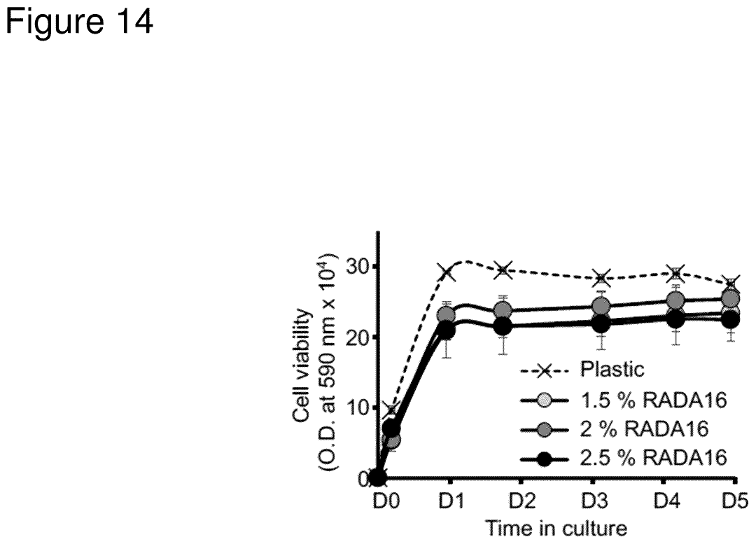

In view of the excellent epidermal regeneration properties of RADA-16 (SEQ ID NO:1), its biocompatibility with the dermis and its ability to interact with NHFs was investigated. . . . First, the ability of RADA-16 (SEQ ID NO:1) to promote human fibroblast adhesion was tested. It was found that RADA-16 (SEQ ID NO:1) enabled NHFs to adhere equally to collagen I and fibronectin, which are known to be important adhesion substrates of fibroblasts. A possible RGD-dependent adhesion mechanism was tested by incubating NHFs with an RGDS peptide (SEQ ID NO:11) to block RGD-dependent integrins (FIG. 13). Although adhesion was not impaired on native collagen I as expected (Taubenberger et al., 2010), it was impaired on fibronectin, which is known to involve RGD-dependent integrins (Ahn et al., 2023), and was also almost completely impaired on RADA-16 (SEQ ID NO:1). These results indicate that adhesion of primary fibroblasts to RADA-16 (SEQ ID NO:1) is RGD-dependent, supporting the hypothesis of RGD biomimicry of the RADA sequence. Monitoring the growth of NHFs cultured in RADA-16 (SEQ ID NO:1) gel using the Alamar assay revealed progressive and regular growth over 5 days, regardless of the concentration of gel tested (FIG. 14). Analysis of cell viability using a LIVE/DEAD assay on longer cultures demonstrated reliable cell growth with only a few dead cells in the gel over a period of 14 and 28 days). Histological analysis of the gels colonized by NHF after 14 and 28 days of culture showed the presence of fibroblasts, which were initially isolated and then accumulated in areas where the gel appeared to have degraded (figure not shown). In sum, these results show that fibroblasts interact with the gel in an RGD-dependent manner and that they can proliferate in the RADA-16 (SEQ ID NO:1) gel.

### REFERENCES

Ahn, S., Sharma, U., Kasuba, K. C., Strohmeyer, N., & Müller, D. J. (2023). Engineered Biomimetic Fibrillar Fibronectin Matrices Regulate Cell Adhesion Initiation, Migration, and Proliferation via a51 Integrin and Syndecan-4 Crosstalk. Advanced Science, 10 (24), 2300812. doi: 10.1002/advs.202300812
Aragona, M., Dekoninck, S., Rulands, S. et al. Defining stem cell dynamics and migration during wound healing in mouse skin epidermis. Nat Commun 8, 14684 (2017). https://doi.org/10.1038/ncomms14684.
Bouatrouss Y, Herring-Gillam F E, Gosselin J, Poisson J, Beaulieu J F. Altered expression of laminins in Crohn's disease small intestinal mucosa. Am J Pathol. 2000 January;156 (1): 45-50. doi: 10.1016/S0002-9440 (10) 64704-9.
Boyce, S. T., Ham, R. G. Cultivation, frozen storage, and clonal growth of normal human epidermal keratinocytes in serum-free media. Journal of Tissue Culture Methods 9, 83-93 (1985). https://doi.org/10.1007/BF01797779
Dave U, Thursz M R, Ebrahim H Y, Burke M M, Townsend E R, Walker M M. Distribution of laminins in the basement membranes of the upper gastrointestinal tract and Barrett's oesophagus. J Pathol. 2004 March;202 (3): 299-304. doi: 10.1002/path.1526.
Decline F, Rousselle P. Keratinocyte migration requires alpha2betal integrin-mediated interaction with the laminin 5 gamma2 chain. J Cell Sci. 2001 February; 114 (Pt 4): 811-23. doi: 10.1242/jcs.114.4.811.
Dos Santos M, Michopoulou A, André-Frei V, Boulesteix S, Guicher C, Dayan G, Whitelock J, Damour O, Rousselle P. Perlecan expression influences the keratin 15-positive cell population fate in the epidermis of aging skin. Aging (Albany NY). 2016 April;8 (4): 751-68. doi: 10.18632/aging.100928
D′Alessio S., L. Gerasi, F. Blasi uPAR-deficient mouse keratinocytes fail to produce EGFR-dependent laminin-5, affecting migration in vivo and in vitro J Cell Sci, 121 (Pt 23) (2008), pp. 3922-3932
Evans M J, Miller L A, Hyde D M. The Basement Membrane Zone in Asthma: The Supracellular Anchoring Network. Curr Respir Med Rev. 2013 August;9 (4): 268-273. doi: 10.2174/1573398x09666131121231852.
Fang W C, Lan C E. The Epidermal Keratinocyte as a Therapeutic Target for Management of Diabetic Wounds. Int J Mol Sci. 2023 Feb. 21;24 (5): 4290. doi: 10.3390/ijms24054290.
Guo M, Toda K, Grinnell F. Activation of human keratinocyte migration on type I collagen and fibronectin. J Cell Sci. 1990 June;96 (Pt 2): 197-205. doi: 10.1242/jcs.96.2.197.
Nguyen B P, Ryan M C, Gil S G, Carter W G. Deposition of laminin 5 in epidermal wounds regulates integrin signaling and adhesion. Curr Opin Cell Biol. 2000 October; 12 (5): 554-62. doi: 10.1016/s0955-0674 (00) 00131-9.
Rousselle P, Aumailley M. Kalinin is more efficient than laminin in promoting adhesion of primary keratinocytes and some other epithelial cells and has a different requirement for integrin receptors. J Cell Biol. 1994 April; 125 (1): 205-14. doi: 10.1083/jcb.125.1.205.
Rousselle P, Beck K. Laminin 332 processing impacts cellular behavior. Cell Adh Migr. 2013 January-Feb;7 (1): 122-34. doi: 10.4161/cam.23132.
Rousselle P, Braye F, Dayan G. Re-epithelialization of adult skin wounds: Cellular mechanisms and therapeutic strategies. Adv Drug Deliv Rev. 2019 June; 146:344-365. doi: 10.1016/j.addr.2018.06.019.
Rousselle P, Keene DR, Ruggiero F, Champliaud MF, Rest M, Burgeson RE. Laminin 5 binds the NC-1 domain of type VII collagen. J Cell Biol. 1997 Aug. 11;138 (3): 719-28. doi: 10.1083/jcb.138.3.719.
Rousselle P, Lunstrum GP, Keene DR, Burgeson RE. Kalinin: an epithelium-specific basement membrane adhesion molecule that is a component of anchoring filaments. J Cell Biol. 1991 August; 114 (3): 567-76. doi: 10.1083/jcb.114.3.567.
Roig-Rosello E, Dayan G, Bovio S, Manissier P, Errazuriz E, Rousselle P. Dermal stiffness governs the topography of the epidermis and the underlying basement membrane in young and old human skin. Aging Cell. 2024 April;23 (4): e14096. doi: 10.1111/acel.14096.
Sasaki T, Fässler R, Hohenester E. Laminin: the crux of basement membrane assembly. J Cell Biol. 2004 Mar. 29;164 (7): 959-63. doi: 10.1083/jcb.200401058.
Simon-Assmann P, Lefebvre O, Bellissent-Waydelich A, Olsen J, Orian-Rousseau V, De Arcangelis A. The laminins: role in intestinal morphogenesis and differentiation. Ann N Y Acad Sci. 1998 Nov. 17; 859:46-64. doi: 10.1111/j.1749-6632.1998.tb11110.x.
Tayem R, Niemann C, Pesch M, Morgner J, Niessen CM, Wickström SA, Aumailley M. Laminin 332 Is Indispensable for Homeostatic Epidermal Differentiation Programs. J Invest Dermatol. 2021 November;141 (11): 2602-2610.e3. doi: 10.1016/j.jid.2021.04.008.
Taubenberger, A. V., Woodruff, M. A., Bai, H., Muller, D. J., & Hutmacher, D. W. (2010). The effect of unlocking RGD-motifs in collagen I on pre-osteoblast adhesion and differentiation. Biomaterials, 31 (10), 2827-2835. Doi: 10.1016/j.biomaterials.2009.12.051
Usui M L, Mansbridge J N, Carter W G, Fujita M, Olerud J E. Keratinocyte migration, proliferation, and differentiation in chronic ulcers from patients with diabetes and normal wounds. J Histochem Cytochem. 2008 July;56 (7): 687-96. doi: 10.1369/jhc.2008.951194.
Watt F M. Role of integrins in regulating epidermal adhesion, growth and differentiation. EMBO J. 2002 Aug. 1;21 (15): 3919-26. doi: 10.1093/emboj/cdf399.
Xue L Y, Zou S M, Zheng S, Liu X Y, Wen P, Yuan Y L, Lin D M, Lu N. Expressions of the γ2 chain of laminin-5 and secreted protein acidic and rich in cysteine in esophageal squamous cell carcinoma and their relation to prognosis. Chin J Cancer. 2011 January;30 (1): 69-78. doi: 10.5732/cjc.010.10071.

A notice is hereby given that the invention is further defined by the claims as granted. One of skill in the art shall be aware of the meaning of the terms used in those claims and recognize the variations they cover, including any art-related equivalents thereof, notwithstanding any amendments that may be desired and/or necessitated to obtain allowance of such claims in any pertinent jurisdiction.

1. A therapeutic or cosmetic composition consisting essentially of:
a) one or more peptides selected from Group (A) consisting of: KADA-4 (SEQ ID NO: 9), RADA-4 (SEQ ID NO:8), RFDA-4 (SEQ ID NO:22), RADF-4 (SEQ ID NO: 25); RADA-8 (SEQ ID NO:6), KADA-8 (SEQ ID NO:7), RFDA-8 (SEQ ID NO: 23), RADF-8 (SEQ ID NO:26), RADA-12 (SEQ ID NO:13); KADA-12 (SEQ ID NO:14), RFDA-12 (SEQ ID NO:24), KADA-16 (SEQ ID NO:5); RFDA-16 (SEQ ID NO:19) and RADF-16 (SEQ ID: 20);
b) two or more peptides selected from Group (B) consisting of: the peptides of Group (A) and RADA-16 (SEQ ID NO:1);
c) two or more peptides selected from Group (C) consisting of: Group (B) and an RGD peptide;
d) one or more peptides selected from Group (D) whose sequence is no more than 50 amino acids long, said sequence comprising any of the peptide sequences recited in Group (A), expressly excluding a fusion peptide comprising the sequence RADA-16 (SEQ ID NO:1) or e) two or more peptides selected from Group (E) consisting of the peptides of Group (B) and Group (D), and
optionally, one or more other active ingredients.

2. The composition of claim 1, wherein the RGD peptide is selected from the group consisting of RGDS (SEQ ID NO:11), RGDYRYDYRYDYRGDY (SEQ ID NO:15), RGDFRFDFRFDFRGDF (SEQ ID NO:16), RGDWRWDWRWDWRGDW (SEQ ID NO: 17), GRGDIPASSKGGGGSRLLLLLLR (SEQ ID NO:18), and GRGDSP (SEQ ID NO: 12).

3. The composition of claim 1, wherein the composition is sterile.

4. The composition of claim 3, wherein the composition has sterility assurance level (SAL) of at least 10⁻⁴ or less.

5. The composition of claim 3, wherein the peptide(s) is/are present in water or aqueous buffer at concentration of 0.1-10% w/v.

6. The composition of claim 3, wherein the composition is formulated for application to wounds or intact skin in a subject.

7. The composition of claim 6, wherein the composition is contained in a pre-filled syringe.

8. The composition of claim 7, wherein the syringe further comprises an applicator for wounds.

9. A method of treating a wound in a mucous membrane, the method comprising administering the composition of claim 3 to a subject, and optionally, re-administering the composition, thereby improving or accelerating the healing of the wound.

10. The method of claim 9, wherein the subject is human.

11. The method of claim 9, wherein the wound is chronic or non-healing.

12. The method of claim 9, wherein the composition is applied via an endoscope to the wound.

13. A method of treating a wound of the skin of a subject, the method comprising administering the composition of claim 3 to a site of the wound of the subject, and optionally, re-administering the composition to the wound, thereby improving or accelerating the healing of the wound.

14. The method of claim 13, wherein the subject is human.

15. The method of claim 13, wherein the composition is applied topically.

16. The method of claim 13, wherein the wound is chronic or non-healing.

17. A method of cosmetically treating a wound or abrasion of a subject, the method comprising administering the composition of claim 3 to the subject, and optionally, re-administering the composition, the method thereby improving the cosmetic appearance of the wound or abrasion.

18. A method of cosmetically treating intact skin, the method comprising applying the composition of claim 3 to the skin of a subject multiple times, thereby improving the appearance of the skin.
